(19) United States
(12) Patent Application Publication
(10) Pub. No.: US 2023/0381329 A1
(43) Pub. Date: Nov. 30, 2023

(54) Zwitterion Polymer-Drug Conjugates

(71) Applicant: University of Utah Research Foundation, Salt Lake City, UT (US)

(72) Inventors: Jessica Kramer, Salt Lake City, UT (US); Danny Hung-Chieh Chou, Palo Alto, CA (US)

(21) Appl. No.: 18/324,773

(22) Filed: May 26, 2023

Related U.S. Application Data
(60) Provisional application No. 63/346,099, filed on May 26, 2022.

(51) Int. Cl. A61K 47/64; A61K 47/69

(52) U.S. Cl. CPC A61K 47/645; A61K 47/6949

(57) ABSTRACT

Disclosed herein, are Zwitterion polymer conjugates. The conjugate comprises a Zwitterion polymer, a linker, and a therapeutic polypeptide. Also described herein, are compositions comprising the conjugates, methods of their preparation, methods of treating diseases with the conjugates or their compositions, and method of preventing aggregation of therapeutic proteins.

### CROSS-REFERENCE TO RELATED APPLICATIONS

This application claims the benefit of the filing date of U.S. Provisional Application No. 63/346,099 which was filed on May 26, 2022. The content of this earlier filed application is hereby incorporated herein by reference in its entirety.

### REFERENCE TO SEQUENCE LISTING

The Sequence Listing submitted herewith as an xml file named “21101_0442U2_Sequence_Listing,” created on May 25, 2023, and having a size of 4,096 bytes is hereby incorporated by reference pursuant to 37 C.F.R. § 1.52(e)(5).

### BACKGROUND

Protein biopharmaceuticals are a rapidly growing area due their advantages in specificity and general biocompatibility. However, a serious challenge encountered in most stages of protein drug development is aggregation. Proteins can aggregate by physical and/or chemical associations through Van der Waals, hydrophobic, or electrostatic forces or by formation of new covalent bonds. Such events typically convert the therapeutic molecules into non-active and potentially toxic substances. This challenge reduces efficacy of on-market drugs and has impeded clinical application of potentially life-saving new drugs. The need remains for a material that is simple and economical to prepare, biodegradable, and based on naturally occurring chemical motifs.

### SUMMARY

Disclosed herein are conjugates, comprising a Zwitterion polymer, a linker, and a therapeutic polypeptide.

Disclosed herein are methods of preventing aggregation of a therapeutic polypeptide, the methods comprising; conjugating a compound to a therapeutic polypeptide, wherein the compound comprises a Zwitterion polymer, wherein the Zwitterion polymer comprises one or more monomer units of S-alkyl-L-methionine sulfonium chloride where the alkyl group contains a carboxylic acid and a linker, wherein the Zwitterion polymer is covalently bonded to linker, and wherein the linker is covalently or non-covalently bonded to the therapeutic polypeptide.

Other features and advantages of the present compositions and methods are illustrated in the description below, the drawings, and the claims.

### BRIEF DESCRIPTION OF THE DRAWINGS

FIG. 1 shows the preparation of oxidized and alkylated PMet structures. Met was converted to Met NCA, which was polymerized to PMet using a Co⁰ catalyst. After end-group functionalization, PMet-Alk was converted to the sulfoxide or the cationic or zwitterionic sulfoniums shown (also referred to as ‘Scheme 1’).

FIG. 2 shows the synthetic scheme to form CB[7]-PMet conjugates via Cu-catalyzed click reaction of azido-CB[7] and alkyne-. PMet. CB [7]-PMet can participate in host-guest complexation with the terminal Phe of human insulin. FIG. 2 is also referred to as ‘scheme 2’) GIVEQCCTSICSLYQLENYCN (SEQ ID NO: 1); and FVNQHLCGSHLVEALYLVCGERGFFYTPKA (SEQ ID NO: 2) are shown.

FIGS. 3A-B show the aggregation of recombinant human (rHu) insulin in combination with various CB[7]-PMet structures. FIG. 3A shows the initial aggregation of rHu insulin alone or with addition of various CB[7]-PMet structures. FIG. 3B shows the aggregation over 100 hours of rHu insulin or rHu insulin with CB[7]-PMet^{CM} of varied chain lengths complexed by host-guest chemistry, or uncomplexed due to lack of CB[7] group.

FIG. 4 shows aqueous circular dichroism analyses of 80-residue PMets, 20° C.

FIGS. 5A-C show stabilizing effects of CB [7]-PMet^{CM}₈₀ on other aggregation-prone proteins. FIG. 5A shows preparation of benzyl-functionalized human calcitonin (hCT) with one (hCT A1) or two (hCT A2) methylene spacers between the terminal amine. FIG. 5B shows that the N-terminal amine was selectively modified on-resin with a benzylic amine group using reductive amination chemistry. FIG. 5C shows aggregation data for hCT, hCT A1, and hCT A2 with and without CB [7]-PMet^{CM}₈₀ where hCT A2 with the zwitterionic polypeptide showed resistance to aggregation. SEQ ID NO: 3 is H2N-CGNLSTCMLGTYTQDFNKFHTFPQTAIGVGAP.

FIGS. 6A-B show the effects of protease susceptibility and cytotoxicity properties of PMet^{CM}₈₀. FIG. 6A show cell viability of MDA-MB-231 cells after treatment with PMet^{CM}₈₀. Data are represented as mean±standard error. **** indicates p<0.0001, and n.s. indicates not significant when compared to PBS control using a Student's t test. FIGS. 6B-D show protease digestion of polypeptide polymer AF350-labeled PMet^{CM}₈₀ visualized on SDS-PAGE gels. FIG. 6B shows the digestion time of 24 hours with enzyme:substrate (E:S) of 1:10 for trypsin and proteinase K and 1:20 for methionine aminopeptidase 2 (METAP2). FIG. 6C shows the digestion time of 48 hours with E:S of 1:1 for proteinase K. FIG. 6D shows the digestion time of 1 week with E:S of 1:1 for papain and proteinase K.

### DETAILED DESCRIPTION

The present disclosure can be understood more readily by reference to the following detailed description of the invention, the figures and the examples included herein.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, the term “at least one of” is intended to be synonymous with “one or more of” For example, “at least one of A, B and C” explicitly includes only A, only B, only C, and combinations of each.

As used herein, the term “subject” refers to the target of administration, e.g., a human. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment for diabetes (type I or type II), such as, for example, prior to the administering step.

As used herein, the term “polymer” refers to a series of monomer groups linked together. In some aspects, polymer refers to a relatively high molecular weight organic compound, natural or synthetic, whose structure can be represented by a repeated small unit, the monomer (e.g., polyethylene, rubber, cellulose). Synthetic polymers are typically formed by addition or condensation polymerization of monomers. In some aspects, the high MW polymers can be prepared from monomers that include, but are not limited to, acrylates, methacrylates, acrylamides, methacrylamides, styrenes, vinyl-pyridine, vinyl-pyrrolidone and vinyl esters such as vinyl acetate. Additional monomers are useful in the high MW polymers of the present invention. When two different monomers are used, the two monomers are called “comonomers” or “copolymer” meaning that the different monomers are copolymerized to form a single polymer. The polymer can be linear or branched. When the polymer is branched, each polymer chain is referred to as a “polymer arm.” The end of the polymer arm linked to the initiator moiety is the proximal end, and the growing-chain end of the polymer arm is the distal end. On the growing chain-end of the polymer arm, the polymer arm end group can be the radical scavenger, or another group. In some aspects, copolymer refers to a polymer formed from two or more different repeating units (monomer residues). By way of example and without limitation, a copolymer can be an alternating copolymer, a random copolymer, a block copolymer, or a graft copolymer. It is also contemplated that, in certain aspects, various block segments of a block copolymer can themselves comprise copolymers.

In various aspects, a polymer or copolymer can be described as the polymerization product of one or more monomers. For example, a copolymer can be described as the product of copolymerization of methionine N-carboxyanhydride and any other amino acid N-carboxyanhydride.

As used herein, the term “reactive group” as used herein refers to a group that is capable of reacting with another chemical group to form a covalent bond, i.e. is covalently reactive under suitable reaction conditions, and generally represents a point of attachment for another substance. The reactive group is a moiety, such as maleimide or succinimidyl ester, on the compounds of the present invention that is capable of chemically reacting with a functional group on a different compound to form a covalent linkage. Reactive groups generally include nucleophiles, electrophiles and photoactivatable groups.

As used herein, the term “functional agent” is defined to include a bioactive agent or a diagnostic agent. A “bioactive agent” is defined to include any agent, drug, compound, or mixture thereof that targets a specific biological location (targeting agent) and/or provides some local or systemic physiological or pharmacologic effect that can be demonstrated in vivo or in vitro. Non-limiting examples include drugs, vaccines, antibodies, antibody fragments, scFvs, diabodies, avimers, vitamins and cofactors, polysaccharides, carbohydrates, steroids, lipids, fats, proteins, peptides, polypeptides, nucleotides, oligonucleotides, polynucleotides, and nucleic acids (e.g., mRNA, tRNA, snRNA, RNAi, DNA, cDNA, antisense constructs, ribozymes, etc). A “diagnostic agent” is defined to include any agent that enables the detection or imaging of a tissue or disease. Examples of diagnostic agents include, but are not limited to, radiolabels, fluorophores and dyes.

As used herein, the term “therapeutic protein” or “therapeutic polypeptide” refers to peptides or proteins that comprise an amino acid sequence which in whole or in part makes up a drug and can be used in human or animal pharmaceutical applications. Numerous therapeutic proteins are known.

“Pharmaceutically acceptable” composition or “pharmaceutical composition” refers to a composition comprising a compound of the invention and a pharmaceutically acceptable excipient or pharmaceutically acceptable excipients.

“Pharmaceutically acceptable excipient” and “pharmaceutically acceptable carrier” refer to an excipient that can be included in the compositions of the invention and that causes no significant adverse toxicological effect on the patient and is approved or approvable by the FDA for therapeutic use, particularly in humans. Non-limiting examples of pharmaceutically acceptable excipients include water, NaCl, normal saline solutions, lactated Ringer's, normal sucrose, normal glucose and the like.

“Therapeutically effective amount” refers to an amount of a conjugated functional agent or of a pharmaceutical composition useful for treating, ameliorating, or preventing an identified disease or condition, or for exhibiting a detectable therapeutic effect. The effect can be detected in an individual patient relative to a baseline measurement before treatment or by determining a statistically significant difference in outcome between treated and control populations.

The “biological half-life” of a substance is a pharmacokinetic parameter which specifies the time required for one half of the substance to be removed from an organism following introduction of the substance into the organism.

As used herein “molecular weight” in the context of the polymer can be expressed as either a number average molecular weight, or a weight average molecular weight or a peak molecular weight. Unless otherwise indicated, all references to molecular weight herein refer to the peak molecular weight. These molecular weight determinations, number average (Mn), weight average (Mw) and peak (Mp), can be measured using size exclusion chromatography or other liquid chromatography techniques. Other methods for measuring molecular weight values can also be used, such as the use of end-group analysis or the measurement of colligative properties (e.g., freezing-point depression, boiling-point elevation, or osmotic pressure) to determine number average molecular weight, or the use of light scattering techniques, ultracentrifugation or viscometry to determine weight average molecular weight. In a preferred embodiment of the present invention, the molecular weight is measured by SEC-MALS (size exclusion chromatography—multi angle light scattering). The polymeric reagents of the invention are typically polydisperse (i.e., number average molecular weight and weight average molecular weight of the polymers are not equal), preferably possessing low polydispersity values of, for example, less than about 1.5, as judged by gel permeation chromatography. In other embodiments, the polydispersities (PDI) are more preferably in the range of about 1.4 to about 1.2, still more preferably less than about 1.15, and still more preferably less than about 1.10, yet still more preferably less than about 1.05, and most preferably less than about 1.03.

As used herein, terms “protected,” “protected form,” “protecting group” and “protective group” refer to the presence of a group (i.e., the protecting group) that prevents or blocks reaction of a particular chemically reactive functional group in a molecule under certain reaction conditions. Protecting groups vary depending upon the type of chemically reactive group being protected as well as the reaction conditions to be employed and the presence of additional reactive or protecting groups in the molecule, if any. Suitable protecting groups include those such as found in the treatise by Greene et al., “Protective Groups In Organic Synthesis,” 3rd Edition, John Wiley and Sons, Inc., New York, 1999.

“Alkyl” refers to a straight or branched, saturated, aliphatic radical having the number of carbon atoms indicated. For example, C1-C6 alkyl includes, but is not limited to, methyl, ethyl, propyl, isopropyl, butyl, isobutyl, sec-butyl, tert-butyl, pentyl, isopentyl, hexyl, etc. Other alkyl groups include, but are not limited to heptyl, octyl, nonyl, decyl, etc. Alkyl can include any number of carbons, such as 1-2, 1-3, 1-4, 1-5, 1-6, 1-7, 1-8, 1-9, 1-10, 2-3, 2-4, 2-2-6, 3-4, 3-5, 3-6, 4-5, 4-6 and 5-6. The alkyl group is typically monovalent, but can be divalent, such as when the alkyl group links two moieties together.

The term “lower” referred to above and hereinafter in connection with organic radicals or compounds respectively defines a compound or radical which can be branched or unbranched with up to and including 7, preferably up to and including 4 and (as unbranched) one or two carbon atoms.

“Alkylene” refers to an alkyl group, as defined above, linking at least two other groups, i.e., a divalent hydrocarbon radical. The two moieties linked to the alkylene can be linked to the same atom or different atoms of the alkylene. For instance, a straight chain alkylene can be the bivalent radical of —(CH2)n, where n is 1, 2, 3, 4, 5 or 6. Alkylene groups include, but are not limited to, methylene, ethylene, propylene, isopropylene, butylene, isobutylene, sec-butylene, pentylene and hexylene.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be a variety of groups selected from: —OR′, ═O, ═NR′, ═N—OR′, —NR′R″, —SR′, -halogen, —SiR′R″R′″, —OC(O)R′, —C(O)R′, —CO2R′, —CONR′R″, —OC(O)NR′R″, —NR″C(O)R′, —NR′—C(O)NR″R′″, —NR″C(O)2R′, —NH—C(NH2)=NH, —NR′ C(NH2)=NH, —NH—C(NH2)=NR′, —S(O)R′, —S(O)2R′, —S(O)2NR′R″, —CN and —NO2 in a number ranging from zero to (2m′+1), where m′ is the total number of carbon atoms in such radical. R′, R″ and R′″ each independently refer to hydrogen, unsubstituted (C1-C8)alkyl and heteroalkyl, unsubstituted aryl, aryl substituted with 1-3 halogens, unsubstituted alkyl, alkoxy or thioalkoxy groups, or aryl-(C1-C4)alkyl groups. When R′ and R″ are attached to the same nitrogen atom, they can be combined with the nitrogen atom to form a 5-, 6-, or 7-membered ring. For example, NR′R″ is meant to include I-pyrrolidinyl and 4-morpholinyl. From the above discussion of substituents, one of skill in the art will understand that the term “alkyl” is meant to include groups such as haloalkyl (e.g., —CF3 and —CH2CF3) and acyl (e.g., —C(O)CH3, —C(O)CF3, —C(O)CH2OCH3, and the like). Preferably, the substituted alkyl and heteroalkyl groups have from 1 to 4 substituents, more preferably 1, 2 or 3 substituents. Exceptions are those perhalo alkyl groups (e.g., pentafluoroethyl and the like) which are also preferred and contemplated by the present invention.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO2R′, —CONR′R″, —OC(O)NR′R″, —NR″C(O)R′, —NR′—C(O)NR″R′″, —NR″C(O)2R′, —NR—C(NR′R″R′″)═NR″″, —NR—C(NR′R″)═NR′″, —S(O)R′, —S(O)2R′, —S(O)2NR′R″, —NRSO2R′, —CN and —NO2 in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

“Alkoxy” refers to alkyl group having an oxygen atom that either connects the alkoxy group to the point of attachment or is linked to two carbons of the alkoxy group. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be further substituted with a variety of substituents described within. For example, the alkoxy groups can be substituted with halogens to form a “halo-alkoxy” group.

“Carboxyalkyl” means an alkyl group (as defined herein) substituted with a carboxy group. The term “carboxycycloalkyl” means a cycloalkyl group (as defined herein) substituted with a carboxy group. The term alkoxyalkyl means an alkyl group (as defined herein) substituted with an alkoxy group. The term “carboxy” employed herein refers to carboxylic acids and their esters.

“Haloalkyl” refers to alkyl as defined above where some or all of the hydrogen atoms are substituted with halogen atoms. Halogen (halo) preferably represents chloro or fluoro, but may also be bromo or iodo. For example, haloalkyl includes trifluoromethyl, fluoromethyl, 1,2,3,4,5-pentafluoro-phenyl, etc. The term “perfluoro” defines a compound or radical which has all available hydrogens that are replaced with fluorine. For example, perfluorophenyl refers to 1,2,3,4,5-pentafluorophenyl, perfluoromethyl refers to 1,1,1-trifluoromethyl, and perfluoromethoxy refers to 1,1,1-trifluoromethoxy.

“Fluoro-substituted alkyl” refers to an alkyl group where one, some, or all hydrogen atoms have been replaced by fluorine.

“Cytokine” in the context of this invention is a member of a group of protein signaling molecules that may participate in cell-cell communication in immune and inflammatory responses. Cytokines are typically small, water-soluble glycoproteins that have a mass of about 8-35 kDa.

“Cycloalkyl” refers to a cyclic hydrocarbon group that contains from about 3 to 12, from 3 to 10, or from 3 to 7 endocyclic carbon atoms. Cycloalkyl groups include fused, bridged and spiro ring structures.

“Endocyclic” refers to an atom or group of atoms which comprise part of a cyclic ring structure.

“Exocyclic” refers to an atom or group of atoms which are attached but do not define the cyclic ring structure.

“Cyclic alkyl ether” refers to a 4 or 5 member cyclic alkyl group having 3 or 4 endocyclic carbon atoms and 1 endocyclic oxygen or sulfur atom (e.g., oxetane, thietane, tetrahydrofuran, tetrahydrothiophene); or a 6 to 7 member cyclic alkyl group having 1 or 2 endocyclic oxygen or sulfur atoms (e.g., tetrahydropyran, 1,3-dioxane, 1,4-dioxane, tetrahydrothiopyran, 1,3-dithiane, 1,4-dithiane, 1,4-oxathiane).

“Alkenyl” refers to either a straight chain or branched hydrocarbon of 2 to 6 carbon atoms, having at least one double bond. Examples of alkenyl groups include, but are not limited to, vinyl, propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can also have from 2 to 3, 2 to 4, 2 to 5, 3 to 4, 3 to 5, 3 to 6, 4 to 4 to 6 and 5 to 6 carbons. The alkenyl group is typically monovalent, but can be divalent, such as when the alkenyl group links two moieties together.

“Alkenylene” refers to an alkenyl group, as defined above, linking at least two other groups, i.e., a divalent hydrocarbon radical. The two moieties linked to the alkenylene can be linked to the same atom or different atoms of the alkenylene. Alkenylene groups include, but are not limited to, ethenylene, propenylene, isopropenylene, butenylene, isobutenylene, sec-butenylene, pentenylene and hexenylene.

“Alkynyl” refers to either a straight chain or branched hydrocarbon of 2 to 6 carbon atoms, having at least one triple bond. Examples of alkynyl groups include, but are not limited to, acetylenyl, propynyl, I-butynyl, 2-butynyl, isobutynyl, sec-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. Alkynyl groups can also have from 2 to 3, 2 to 4, 2 to 5, 3 to 4, 3 to 5, 3 to 6, 4 to 4 to 6 and 5 to 6 carbons. The alkynyl group is typically monovalent, but can be divalent, such as when the alkynyl group links two moieties together.

“Alkynylene” refers to an alkynyl group, as defined above, linking at least two other groups, i.e., a divalent hydrocarbon radical. The two moieties linked to the alkynylene can be linked to the same atom or different atoms of the alkynylene. Alkynylene groups include, but are not limited to, ethynylene, propynylene, butynylene, sec-butynylene, pentynylene and hexynylene.

“Cycloalkyl” refers to a saturated or partially unsaturated, monocyclic, fused bicyclic or bridged polycyclic ring assembly containing from 3 to 12 ring atoms, or the number of atoms indicated. Monocyclic rings include, for example, cyclopropyl, cyclobutyl, cyclopentyl, cyclohexyl, and cyclooctyl. Bicyclic and polycyclic rings include, for example, norbornane, decahydronaphthalene and adamantane. For example, C3-8cycloalkyl includes cyclopropyl, cyclobutyl, cyclopentyl, cyclohexyl, cyclooctyl, and norbornane.

“Cycloalkylene” refers to a cycloalkyl group, as defined above, linking at least two other groups, i.e., a divalent hydrocarbon radical. The two moieties linked to the cycloalkylene can be linked to the same atom or different atoms of the cycloalkylene. Cycloalkylene groups include, but are not limited to, cyclopropylene, cyclobutylene, cyclopentylene, cyclohexylene, and cyclooctylene.

“Heterocycloalkyl” refers to a ring system having from 3 ring members to about 20 ring members and from 1 to about 5 heteroatoms such as N, O and S. Additional heteroatoms can also be useful, including, but not limited to, B, Al, Si and P. The heteroatoms can also be oxidized, such as, but not limited to, —S(O)— and —S(O)₂—. For example, heterocycle includes, but is not limited to, tetrahydrofuranyl, tetrahydrothiophenyl, morpholino, pyrrolidinyl, pyrrolinyl, imidazolidinyl, imidazolinyl, pyrazolidinyl, pyrazolinyl, piperazinyl, piperidinyl, indolinyl, quinuclidinyl and 1,4-dioxa-8-aza-spiro[4.5]dec-8-yl.

“Heterocycloalkylene” refers to a heterocycloalkyl group, as defined above, linking at least two other groups. The two moieties linked to the heterocycloalkylene can be linked to the same atom or different atoms of the heterocycloalkylene.

“Aryl” refers to a monocyclic or fused bicyclic, tricyclic or greater, aromatic ring assembly containing 6 to 16 ring carbon atoms. For example, aryl may be phenyl, benzyl or naphthyl, preferably phenyl. “Arylene” means a divalent radical derived from an aryl group. Aryl groups can be mono-, di- or tri-substituted by one, two or three radicals selected from alkyl, alkoxy, aryl, hydroxy, halogen, cyano, amino, amino-alkyl, trifluoromethyl, alkylenedioxy and oxy-C2-C3-alkylene; all of which are optionally further substituted, for instance as hereinbefore defined; or 1- or 2-naphthyl; or 1- or 2-phenanthrenyl. Alkylenedioxy is a divalent substitute attached to two adjacent carbon atoms of phenyl, e.g. methylenedioxy or ethylenedioxy. Oxy-C2-C3-alkylene is also a divalent substituent attached to two adjacent carbon atoms of phenyl, e.g. oxyethylene or oxypropylene. An example for oxy-C2-C3-alkylene-phenyl is 2,3-dihydrobenzofuran-5-yl.

In some aspects, an aryl is naphthyl, phenyl or phenyl mono- or disubstituted by alkoxy, phenyl, halogen, alkyl or trifluoromethyl, phenyl or phenyl-mono- or disubstituted by alkoxy, halogen or trifluoromethyl.

Examples of substituted phenyl groups as R are, e.g. 4-chlorophen-1-yl, 3,4-dichlorophen-1-yl, 4-methoxyphen-1-yl, 4-methylphen-1-yl, 4-aminomethylphen-1-yl, 4-methoxyethylaminomethylphen-1-yl, 4-hydroxyethylaminomethylphen-1-yl, 4-hydroxyethyl-(methyl)-aminomethylphen-1-yl, 3-aminomethylphen-1-yl, 4-N-acetylaminomethylphen-1-yl, 4-aminophen-1-yl, 3-aminophen-1-yl, 2-aminophen-1-yl, 4-phenyl-phen-1-yl, 4-(imidazol-1-yl)-phenyl, 4-(imidazol-1-ylmethyl)-phen-1-yl, 4-(morpholin-1-yl)-phen-1-yl, 4-(morpholin-1-ylmethyl)-phen-1-yl, 4-(2-methoxyethylaminomethyl)-phen-1-yl and 4-(pyrrolidin-1-ylmethyl)-phen-1-yl, 4-(thiophenyl)-phen-1-yl, 4-(3-thiophenyl)-phen-1-yl, 4-(4-methylpiperazin-1-yl)-phen-1-yl, and 4-(piperidinyl)-phenyl and 4-(pyridinyl)-phenyl optionally substituted in the heterocyclic ring.

“Arylene” refers to an aryl group, as defined herein, linking at least two other groups. The two moieties linked to the arylene are linked to different atoms of the arylene. Arylene groups include, but are not limited to, phenylene.

“Arylene-oxy” refers to an arylene group, as defined above, where one of the moieties linked to the arylene is linked through an oxygen atom. Arylene-oxy groups include, but are not limited to, phenylene-oxy.

Similarly, substituents for the aryl and heteroaryl groups are varied and are selected from: -halogen, —OR′, —OC(O)R′, —NR′R″, —SR′, —R′, —CN, —NO2, —CO2R′, —CONR′R″, —C(O)R′, —OC(O)NR′R″, —NR″C(O)R′, —NR″C(O)2R′, —NR′—C(O)NR″R′″, —NH—C(NH2)=NH, —NR′C(NH2)=NH, —NH—C(NH2)=NR′, —S(O)R′, —S(O)2R′, —S(O)2NR′R″, —N3, —CH(Ph)2, perfluoro(C1-C4)alkoxy, and perfluoro(C1-C4)alkyl, in a number ranging from zero to the total number of open valences on the aromatic ring system; and where R′, R″ and R′″ are independently selected from hydrogen, (C1-C8)alkyl and heteroalkyl, unsubstituted aryl and heteroaryl, (unsubstituted aryl)-(C1-C4)alkyl, and (unsubstituted aryl)oxy-(C1-C4)alkyl.

Two of the substituents on adjacent atoms of the aryl or heteroaryl ring may optionally be replaced with a substituent of the formula -T-C(O)—(CH₂)q-U—, wherein T and U are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula -A-(CH2)r-B—, wherein A and B are independently —CH₂—, —O—, —NH—, —S—, —S(O)—, —S(O)₂—, S(O)2NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)s-X—(CH₂)t-, where s and t are independently integers of from 0 to 3, and X is —O—, —NR′—, —S—, —S(O)—, —S(O)₂—, or S(O)₂NR′—. The substituent R′ in —NR′— and —S(O)₂NR′— is selected from hydrogen or unsubstituted (C1-C6)alkyl.

“Heteroaryl” refers to a monocyclic or fused bicyclic or tricyclic aromatic ring assembly containing 5 to 16 ring atoms, where from 1 to 4 of the ring atoms are a heteroatom each N, O or S. For example, heteroaryl includes pyridyl, indolyl, indazolyl, quinoxalinyl, quinolinyl, isoquinolinyl, benzothienyl, benzofuranyl, furanyl, pyrrolyl, thiazolyl, benzothiazolyl, oxazolyl, isoxazolyl, triazolyl, tetrazolyl, pyrazolyl, imidazolyl, thienyl, or any other radicals substituted, especially mono- or di-substituted, by e.g. alkyl, nitro or halogen. Pyridyl represents 2-, 3- or 4-pyridyl, advantageously 2- or 3-pyridyl. Thienyl represents 2- or 3-thienyl. Quinolinyl represents preferably 2-, 3- or 4-quinolinyl. Isoquinolinyl represents preferably 1-, 3- or 4-isoquinolinyl. Benzopyranyl, benzothiopyranyl represents preferably 3-benzopyranyl or 3-benzothiopyranyl, respectively. Thiazolyl represents preferably 2- or 4-thiazolyl, and most preferred, 4-thiazolyl. Triazolyl is preferably 1-, 2- or 5-(1,2,4-triazolyl). Tetrazolyl is preferably 5-tetrazolyl.

In some aspects, heteroaryl can be pyridyl, indolyl, quinolinyl, pyrrolyl, thiazolyl, isoxazolyl, triazolyl, tetrazolyl, pyrazolyl, imidazolyl, thienyl, furanyl, benzothiazolyl, benzofuranyl, isoquinolinyl, benzothienyl, oxazolyl, indazolyl, or any of the radicals substituted, especially mono- or di-substituted.

As used herein, the term “heteroalkyl” refers to an alkyl group having from 1 to 3 heteroatoms such as N, O and S. Additional heteroatoms can also be useful, including, but not limited to, B, Al, Si and P. The heteroatoms can also be oxidized, such as, but not limited to, —S(O)— and —S(O)₂—. For example, heteroalkyl can include ethers, thioethers, alkyl-amines and alkyl-thiols.

As used herein, the term “heteroalkylene” refers to a heteroalkyl group, as defined above, linking at least two other groups. The two moieties linked to the heteroalkylene can be linked to the same atom or different atoms of the heteroalkylene.

“Electrophile” refers to an ion or atom or collection of atoms, which may be ionic, having an electrophilic center, i.e., a center that is electron seeking, capable of reacting with a nucleophile. An electrophile (or electrophilic reagent) is a reagent that forms a bond to its reaction partner (the nucleophile) by accepting both bonding electrons from that reaction partner.

“Nucleophile” refers to an ion or atom or collection of atoms, which may be ionic, having a nucleophilic center, i.e., a center that is seeking an electrophilic center or capable of reacting with an electrophile. A nucleophile (or nucleophilic reagent) is a reagent that forms a bond to its reaction partner (the electrophile) by donating both bonding electrons. A “nucleophilic group” refers to a nucleophile after it has reacted with a reactive group. Non limiting examples include amino, hydroxyl, alkoxy, haloalkoxy and the like.

For the purpose of this disclosure, “naturally occurring amino acids” found in proteins and polypeptides are L-alanine, L-arginine, L-asparagine, L-aspartic acid, L-cysteine, L-glutamine, L-glutamic acid, L-glycine, L-histidine, L-isoleucine, L-leucine, L-lysine, L-methionine, L-phenylalanine, L-proline, L-serine, L-threonine, L-tryptophan, L-tyro sine, and or L-valine. “Non-naturally occurring amino acids” found in proteins are any amino acid other than those recited as naturally occurring amino acids. Non-naturally occurring amino acids include, without limitation, the D isomers of the naturally occurring amino acids, and mixtures of D and L isomers of the naturally occurring amino acids. Other amino acids, such as 4-hydroxyproline, desmosine, isodesmosine, 5-hydroxylysine, epsilon-N-methyllysine, 3-methylhistidine, although found in naturally occurring proteins, are considered to be non-naturally occurring amino acids found in proteins for the purpose of this disclosure as they are generally introduced by means other than ribosomal translation of mRNA.

“Linear” in reference to the geometry, architecture or overall structure of a polymer, refers to polymer having a single polymer arm.

“Branched,” in reference to the geometry, architecture or overall structure of a polymer, refers to a polymer having 2 or more polymer “arms” extending from a core structure contained within an initiator. The initiator may be employed in an atom transfer radical polymerization (ATRP) reaction. A branched polymer may possess 2 polymer chains (arms), 3 polymer arms, 4 polymer arms, 5 polymer arms, 6 polymer arms, 7 polymer arms, 8 polymer arms, 9 polymer arms or more. Each polymer arm extends from a polymer initiation site. Each polymer initiation site is capable of being a site for the growth of a polymer chain by the addition of monomers. For example and not by way of limitation, using ATRP, the site of polymer initiation on an initiator is typically an organic halide undergoing a reversible redox process catalyzed by a transition metal compound such as cuprous halide. Preferably, the halide is a bromine.

Insulin is one such example therapeutic that can undergo both physical and chemical aggregation. Insulin is a 51-residue protein that has been the cornerstone of diabetes treatment for close to a century. Through physical associations and covalent bond formation, monomeric insulin can form soluble hexamers and dimers, as well as insoluble fibrils, aggregates, and covalent polymeric species. Insoluble species present a major hurdle since they are no longer therapeutically active. Such aggregation events are exacerbated by mechanical agitation in solution, which is a routine occurrence during shipping and transport. Therefore, the development of novel insulin formulations with enhanced stability and without the need for continuous cold-chain delivery are highly desired.

The use of neutral, hydrophilic polymers has been investigated as a strategy reduce or inhibit the aggregation of therapeutic proteins and peptides. For example, the covalent modification of protein therapeutics with polyethylene glycol (PEG), an approach known as PEGylation, is a widely explored method to increase the stability and circulation half-life of biopharmaceuticals, including for anti-aggregation applications with insulin. However, an emerging body of literature has demonstrated the presence of anti-PEG antibodies in response to PEGylated therapeutics, and has suggested this modification is associated with increased immunogenicity and drug clearance. Additionally, PEG is not biodegradable and can accumulate intracellularly.

An approach that has been proposed to circumvent some of these challenges is supramolecular PEGylation, which is the modification of proteins with PEG chains through non-covalent interactions. One such approach demonstrated PEG conjugated to a cucurbit[7]uril (CB [7]) macrocycle, which preferentially recognizes and binds the N-terminal phenylalanine residue on insulin (Kₐ=1.5×10⁶ M⁻¹), as a dynamic non-covalent route to stabilize insulin formulations. Covalent modifications of insulin at this site are not typically associated with a loss of activity, and routes for supramolecular formulation of insulin with CB [7]-PEG likewise demonstrated activity of aged insulin that was indistinguishable from freshly dissolved insulin. It has been demonstrated that binding of CB [7] to N-terminal aromatic residues arising from the inclusion of the R-group as a guest within the cavity of the CB [7] macrocycle in concert with electrostatic interactions between the protonated N-terminal amino group and the electronegative carbonyl-lined portal. The affinity and rapid exchange dynamics of the insulin-CB [7] interaction ensures the complex remains bound at formulation concentrations in a vial but rapidly dissociates once diluted in the body. Accordingly, this approach might reduce the risks associated with covalent PEGylation by separating the presentation of PEG from the therapeutic. This approach has thus been used to stabilize fast-acting insulin monomers and insulin-pramlintide co-formulations. The affinity for supramolecular recognition can also be tuned by inclusion of higher affinity non-natural guests to afford extended pharmacokinetics.

The growing issues surrounding the use of PEG still demonstrate a clear need for an expanded toolbox of biocompatible and relatively inert polymers for use in protein formulation, nanomedicine, biomedical device coatings, and other applications that are thus far dominated by PEG. Hydrophilic, neutral, saccharide-bearing structures have been explored as protein stabilizers and as PEG alternatives. For example, glycosaminoglycans were covalently conjugated to insulin via non-specific N-hydroxysuccinimide (NHS)-ester/maleimide chemistry. The conjugation site was found to affect activity and the conjugates had variable efficacy in rodent models. Saccharide functionalized methacrylate has also been conjugated to an insulin lysine residue, but these scaffolds are not biodegradable. More recently, hydrolyzable scaffolds were explored using polylactide backbones appended with saccharides and zwitterionic ammonium betaines. Insulin aggregation upon shaking was mitigated when the solution was supplemented with a 10-fold excess of the polymer.

Similar to saccharides and PEG, zwitterionic structures have advantageous properties in that they are hydrophilic but overall neutral. Methacrylate-based sulfobetaine nanogels were shown to inhibit formation of toxic lysozyme nanofibrils. Despite these collective efforts, the need remains for a material that is simple and economical to prepare, active in low concentration, biodegradable, and based on naturally occurring chemical motifs.

A series of hydrophilic, neutral or zwitterionic polypeptide structures have been reported based on the naturally occurring amino acid methionine (Met). PolyMet (PMet) is attractive for biomedical applications because it can be prepared via a simple, scalable, and low-cost polymerization reaction. Further, Met itself is a relatively economical amino acid since it is used in livestock feed. Compared to PEG and other previously explored materials, PMet is particularly appealing since it can degrade into a naturally occurring amino acid.

Met's thioether moiety can be oxidized to the sulfoxide or sulfone forms, or alkylated with a variety of electrophiles to generate stable sulfonium salts. Sulfonium salts of Met are naturally occurring in foods and in cellular methylation processes. Alkylation with a carboxymethyl group (Met^{CM}) yields a zwitterionic structure. Such alkylation reactions are simple, quantitative, can be performed in organic and aqueous solvents, and are chemoselective in peptides and proteins at low pH since Met nucleophilicity is not dependent upon protonation. Oxidation of Met to Met sulfoxide (Met^{O}) in proteins is well-known and the reaction is believed to scavenge reactive oxygen species. Met likely serves to prevent irreversible oxidation at other important residues since Met^{O} can be reduced back to Met with natural enzymes. Further, polymers of Met^{O} have dimethylsulfoxide (DMSO)-like solubilizing properties and are reported to be nontoxic at 2.0 g/kg when administered intravenously to mice. PMet^{O} is readily prepared from PMet by simple treatment with hydrogen peroxide. Due to the ease of preparation, the results provided herein assess the anti-aggregation properties of these neutral, hydrophilic, PMet structures based on naturally occurring building blocks.

To capture the beneficial properties of supramolecular protein formulation, a single CB[7] macrocycle was appended as a terminal group to different PMets, enabling evaluation of this approach for protein stabilization as a formulation additive. This strategy resulted in very high insulin stability, with no aggregation even upon continuous agitation for >100 hours. Thus, the results demonstrated the ability to capture the functional benefits of PEG using a biodegradable, non-cytotoxic polymer based on natural amino acid motifs as a formulation additive to inhibit protein aggregation.

Described herein are zwitterionic polypeptides that were prepared a via rapid and scalable polymerization technique for their ability to inhibit the aggregation of protein therapeutics. The polypeptides are based on the amino acid methionine, and various chain lengths of zwitterion sulfoniums were compared. The results show that zwitterionic structures of sufficient chain lengths were highly efficient inhibitors of therapeutic protein aggregation. The anti-aggregant polypeptides exhibited no cytotoxicity in human cells even at a 5-fold excess of the intended therapeutic regime. The treatment of the Zwitterionic polypeptides were also examined with a panel of natural proteases and it was found that they are slowly biodegradable, which indicates they can be used to provide an improvement in circulation time in addition to anti-aggregation properties.

Described herein are various functional groups that can be included onto polypeptides by alkylation of thioether (a.k.a. sulfide) groups. The thioether groups can be present in the polypeptides, or added to polypeptides containing thioether precursors, such as thiol, alkene or alkyl halide functional groups.

In some aspects, the modification of polypeptides via the thioether groups naturally present in methionine or in S-alkyl cysteine residues. In some aspects, chemically reactive functionalities can be added to polypeptides via this process, including but not limited to alkenes, alkynes, boronic acids, sulfonates, phosphonates, alkoxysilanes, carbohydrates, secondary, tertiary, quaternary and alkylated amines, pyridines, alkyl halides, and ketones, creating functional polypeptides. In some aspects, the chemically reactive functionalities that can be added to polypeptides via this process include but are not limited to amine, thiol, carboxylic acid, allyl group, or one of many bioorthogonal groups. In some aspects, conjugation chemistry can be an amine, thiol, carboxylic acid, hydroxyl, allyl, alkyne, azide, tetrazine, cyclooctyne, aminooxy, phosphine, or cyclopropane.

This alkylation process is chemically selective, allowing to introduce chemically reactive functionality to specific locations on polypeptides, peptides, and proteins. The disclosed polypeptides with complex functionality can be used in applications including but not limited to therapeutics, diagnostics, antimicrobials, delivery vehicles, coatings, composites, and regenerative medicine.

The process of preparing reactive and functional polypeptide compositions as can be carried out by attending to a variety of parameters including but not limited to nature of the alkylating agent, polypeptide composition (percentage of methionine in the polymers, peptide or proteins), use of other thioether containing polypeptides (e.g. S-alkyl cysteines), polypeptide architecture (block or random), use of D- or L-amino acids in the polymers, and conjugation of the polypeptide segments to other synthetic polymers. In some aspects, functionality can be added to the thioether groups found in other synthetic polymers, as these functional groups are readily created from widely used thiol-ene conjugation reactions. In some aspects, other alkylating agents or alkylation processes can be used to create similar functionalized polypeptides. For example, other XCH₂C(O)R reagents, where X=Br or I; other benzylic/pseudo-benzylic bromides, iodides, or triflates; alkyl triflates of the general structure RCH₂CH₂OTf (typically prepared from commonly available (RCH₂CH₂OH); alkyl bromides or iodides of the general structure RCH₂CH₂X, where X=Br or I, (R=functional or reactive residue) can be used.

PCT/US2013/033938 is incorporated herein by reference for its disclosure of preparing functionalized polypeptides and proteins.

Conjugates

Disclosed herein are conjugates that prevent aggregation of a therapeutic polypeptide or protein. In some aspects, the conjugates can be administered to a subject to a patient in need thereof.

In some aspects, the conjugates can comprise a Zwitterion polymer, a linker, and a therapeutic polypeptide. In some aspects, the linker can be cucurbit[n]uril. In some aspects, the conjugates can comprise a Zwitterion polymer, cucurbit[n]uril, and a therapeutic polypeptide.

In some aspects, the Zwitterion polymer can comprise one or more monomer units of S-alkyl-L-methionine sulfonium chloride where the alkyl group contains a carboxylic acid. In some aspects, the S-alkyl-L-methionine sulfonium chloride where the alkyl group contains a carboxylic acid can be alkylated or oxidized.

In some aspects, the Zwitterion polymer can be covalently bonded to a linker. In some aspects, the linker can be covalently bonded to the therapeutic polypeptide. In some aspects, the linker is not covalently bonded to the therapeutic polypeptide. In some aspects, the linker can be covalently bonded to an amino group, a hydroxyl group, a sulfhydryl group or a carboxyl group of the therapeutic polypeptide.

In some aspects, the Zwitterion polymer can be covalently bonded to cucurbit[n]uril. In some aspects, the cucurbit[n]uril is not covalently bonded to the therapeutic polypeptide. In some aspects, the cucurbit[n]uril can be non-covalently bonded to an amino group, a hydroxyl group, a sulfhydryl group or a carboxyl group of the therapeutic polypeptide. In some aspects, the cucurbit[n]uril can be cucurbit[n]uril. In some aspects, n can be 5, 6, 7, or 8.

In some aspects, the Zwitterion polymer portion of the conjugate can have a peak molecular weight between 2000 and 80,000 daltons.

A wide variety of therapeutic peptides or polypeptides can be incorporated into the disclosed conjugates. A therapeutic peptide can be any polypeptide that acts as a hormone, growth factor, neurotransmitter, ion channel ligand, or anti-infective agent.

Examples of therapeutic polypeptides include, but are not limited to insulin, oxytocin, vasopressin, and gonadotropin-releasing hormone (GnRH), Trulicity (dulaglutide), Victoza (liraglutide), Ozempic (semaglutide), Exenatide, Liraglutide, Lixisenatide, Albiglutide, Dulaglutide, Semaglutide, Teduglutide, Linaclotide, Pramlintide, Abarelix, Degarelix, Carfilzomib, Mifamurtide, Aviptadil, Atosiban, Carbetocin, Taltirelin, Bremelanotide, Teriparatide, Abaloparatide, Plecanatide, Nesiritide, Angiotensin II, Icatibant, Enfuvirtide, Tesamorelin, Ziconotide, Romiplostim, Peginesatide, Lucinactant, Etelcalcetide, Afamelanotide, Pasireotide, Lutetium Lu 177 dotatate, Edotreotide gallium Ga-68, or Setmelanotide.

In a further aspect, the therapeutic polypeptide can be an antibody. As used herein, the term “antibody” means a protein made by plasma cells in response to an antigen that typically consist of four subunits including two heavy chains and two light chains. Examples of antibodies include, but are not limited to, bevacizumab, trastuzumab, rituximab, abciximab, adalimumab, alemtuzumab, basiliximab, belimumab, brentuximab vedotin, canakinumab, cetuximab, certolizumab pegol, daclizumab, denosumab, eculizumab, efalizumab, gemtuzumab, golimumab, ibritumomab tiuxetan, infliximab, ipilimumab, muromonab-CD3, natalizumab, ofatumumab, omalizumab, palivizumab, panitumumab, ranibizumab, raxibacumab, tocilizumab, tositumomab and ustekinumab. Other examples of antibodies include, but are not limited to, 3F8, abagovomab, abatacept, acz885, adecatumumab, afelimomab, aflibercept, afutuzumab, alacizumab, altumomab, anatumomab, anrukinzumab, apolizumab, arcitumomab, aselizumab, atlizumab, atorolimumab, bapineuzumab, bavituximab, bectumomab, belatacept, bertilimumab, besilesomab, biciromab, bivatuzumab, blinatumomab, cantuzumab, capromab, catumaxomab, cedelizumab, citatuzumab, cixutumumab, clenoliximab, cnto1275(=ustekinumab), cnto148(=golimumab), conatumumab, dacetuzumab, detumomab, dorlimomab, dorlixizumab, ecromeximab, edobacomab, edrecolomab, efungumab, elsilimomab, enlimomab, epitumomab, epratuzumab, erlizumab, ertumaxomab, etanercept, etaracizumab, exbivirumab, fanolesomab, faralimomab, felvizumab, figitumumab, fontolizumab, foravirumab, galiximab, gantenerumab, gavilimomab, gomiliximab, ibalizumab, igovomab, imciromab, inolimomab, inotuzumab ozogamicin, iratumumab, keliximab, labetuzumab, lebrilizumab, lemalesomab, lerdelimumab, lexatumumab, libivirurnab, lintuzumab, lucatumumab, lumiliximab, mapatumumab, maslimomab, matuzumab, mepolizumab, metelimumab, milatuzumab, minretumomab, mitumomab, morolimumab, motavizumab, myo-029, nacolomab, naptumomab, nebacumab, necitumumab, nerelimomab, nimotuzumab, nofetumomab, ocrelizumab, odulimomab, oportuzumab, oregovomab, otelixizumab, pagibaximab, panobacumab, pascolizumab, pemtumomab, pertuzumab, pexelizumab, pintumomab, priliximab, pritumumab, pro-140, rafivirumab, ramucirumab, regavirumab, reslizumab, rilonacept, robatumumab, rovelizumab, rozrolimupab, ruplizumab, satumomab, sevirumab, sibrotuzumab, siltuximab, siplizumab, solanezumab, sonepcizumab, sontuzumab, stamulurnab, sulesomab, tacatuzumab, tadocizumab, talizumab, tanezumab, tapliturnomab, tefibazumab, telimomab, tenatumomab, teneliximab, teplizumab, tgn1412, ticilimumab (=tremelimumab), tigatuzumab, tnx-355 (=ibalizumab), tnx-650, tnx-901 (=talizumab), toralizumab, tremelimumab, tucotuzumab, tuvirumab, urtoxazumab, vapaliximab, vedolizumab, veltuzumab, vepalimomab, visilizumab, volociximab, votumumab, zalutumumab, zanolimumab, ziralimumab, and zolimomab.

In a further aspect, the therapeutic polypeptide can be an antibody fragment. As used herein, the term “antibody fragment” means a component derived from antigen-specific fragments of antibodies produced by recombinant processes. Three general types of fragments were observed, antigen-binding fragments (Fab), single chain variable fragments (scFv) and “third generation” (3G). Examples of antibody fragments include, but are not limited to, anti-HER2 scFv, Fv, Fab, Fab′, F(ab′)2, Fab′-SH, and scFv.

In a further aspect, the therapeutic polypeptide can be an aptamer. As used herein, the term “aptamer” means an oligonucleotide or peptide molecule that binds to a specific target molecule. Examples of aptamers include, but are not limited to, EpCAM aptamer, nucleic acid aptamers (e.g., DNA aptamers and RNA aptamers) and peptide aptamers.

In a further aspect, the therapeutic polypeptide is a non-antibody protein. As used herein, the term “non-antibody protein” means a large molecule composed of one or more chains of amino acids in a specific order that is not an antibody as defined herein above. Examples of non-antibody proteins include, but are not limited to, albumin, insulin, receptors, actin, and tubulin.

In a further aspect, the therapeutic polypeptide is a peptide. As used herein, the term “peptide” means a molecule consisting of from about 2 to about 50 amino acids. Examples of peptides include, but are not limited to, somatostatin peptide, luteinizing hormone releasing hormone, fusion proteins, receptors, ligands of cell surface proteins, secreted proteins, and enzymes.

Typically any therapeutic that can be covalently bound to the Zwitterion polymer can be used. The therapeutic peptide or polypeptide can be a chemical compound (e.g., peptide) or a protein. In some aspects, the therapeutic can be any therapeutic that is prone to aggregation. In some aspects, the therapeutic polypeptide can be insulin, calcitonin, erythropoietin, or analogs thereof or combinations thereof. In some aspects, the therapeutic polypeptide can be an antibody. In some aspects, the therapeutic polypeptide can be a chimeric antibody. Examples of chimeric antibodies include but are not limited to regdanvimab, trastuzumab, pertuzumab, bevacizumab, rituximab, adalimumab, and Etanercept. Nucleophilic groups on proteins, including antibodies, which can be used to conjugate polymer in accordance with an aspect of the present invention include, but are not limited to: (i)N-terminal amine groups, (ii) side chain amine groups, e.g. lysine, (iii) side chain thiol groups, e.g. cysteine, and (iv) sugar hydroxyl or amino groups where the protein is glycosylated. Amine, thiol, and hydroxyl groups are nucleophilic and capable of reacting to form covalent bonds with electrophilic groups on linker moieties and linker reagents attached to the polymer including: (i) active esters such as NHS esters. HOBt esters, haloformates, and acid halides; (ii) alkyl and benzyl halides such as haloacetamides; (iii) aldehydes, ketones, carboxyl, and maleimide groups. Many proteins, including antibodies, have cysteine thiol groups which can potentially be used for conjugation. Many cysteine residues are in the form of reducible interchain disulfides, i.e. cysteine bridges. Cysteine residues in the form of disulfides are generally not available to react with reagents such as maleimide. Cysteine residues may also be free or unpaired. However, free cysteine residues are frequently found to be “capped” by one or more reagents in various media and are also not available for conjugation. Cysteine residues may be made reactive for conjugation with linker reagents such as maleimide by treatment with a reducing agent such as DTT (dithiothreitol) or tricarbonylethylphosphine (TCEP), such that the protein is fully or partially reduced. Each cysteine bridge will thus form, theoretically, two reactive thiol nucleophiles. In the case of free cysteine, one thiol nucleophile is formed by reduction. Depending cm the conditions employed, reduction by TCEP or DTT can result in the loss of proper protein folding with concomitant loss of activity. However, activity may be recovered by allowing protein refolding under the appropriate conditions.

Various other therapeutic peptides or polypeptides, as will be recognized by one skilled in the art, can also be employed in the present conjugates.

In some aspects, the linker can be a chemical moiety that links two groups together. The linker can be cleavable or non-cleavable. Cleavable linkers can be hydrolyzable, enzymatically cleavable, pH sensitive, photolabile, or disulfide linkers, among others. Other linkers include homobifunctional and heterobifunctional linkers. A linker can be a polypeptide linker, a nucleic acid linker or chemical linker. A “linking group” is a functional group capable of forming a covalent linkage consisting of one or more bonds to a bioactive agent. A linker can also refer to a bifunctional traceless linker that can be used, for example, to temporarily attach highly solubilizing peptide sequences (e.g., sequences of lysine residues) onto a peptide (e.g., an insoluble peptide). See, e.g., Jacobsen et al. (2016) JACS 138: 11775-11782.

Examples of linkers include, but are not limited to, polyethers, small aryl groups (e.g., 1,4-linked benzyl), disulfides, ethers, thioethers, esters, sulfonamides, dipeptides, maleimidocaproyl, hydrazines, hydrazones, acylhydrazines, acylhydrazones, and 1,2,3-triazoles. Desirable qualities of the linker include, but are not limited to, providing stability prior to entering a target cell, providing efficient payload release once inside the target cell (e.g., via endosomal or lysosomal degradation), and compatibility with the disclosed compositions.

In a further aspect, the linker can be cleavable (i.e., the linker relies on the physiological environment and releases a payload via hydrolyzation or proteolysis in the target cells). Examples of cleavable linkers include, but are not limited to, chemically labile linkers (i.e., acid cleavable linkers such as hydrazines and silyl ethers and reducible linkers) and enzyme cleavable linkers (i.e., linkers that rely on the presence of hydrolytic enzymes in the cell). Enzyme cleavable linkers include, but are not limited to, peptide-based linkers (e.g., valine-citrulline) dipeptide linkers and phenylalanine-lysine dipeptide linkers) and beta-glucuronide linkers. In various aspects, a cleavable linker is broken down in the cells to release a compound.

In a further aspect, the linker can be non-cleavable (i.e., the linker cannot be broken down outside a target cell). Advantages of a non-cleavable linker include, but are not limited to, increased plasma stability and larger therapeutic windows. In various aspects, a non-cleavable linker remains attached to a compound in cells.

In a further aspect, the linker can be a tertiary amine linker (e.g., monomethyl auristatin E).

In some aspects, the linker can comprise an alkyl or aryl group with or without oxygen, sulfur, or nitrogen atoms.

A “polypeptide linker” is a polypeptide comprising two or more amino acid residues joined by peptide bonds that are used to link two polypeptides (e.g., a VH and VL domain or a VH domain and an extracellular trap segment). Examples of such linker polypeptides are well known in the art (see, e.g., Holliger et al. (1993) Proc. Natl. Acad. Sci. USA 90:6444-6448; Poljak et al. (1994) Structure 2:1121-1123).

In a further aspect, the linker can be a disulfide linker. Examples of disulfide linkers include, but are not limited to:

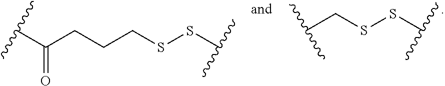 [SMILES: *CSS* ; *SSCCCC(*)=O]

In a further aspect, the linker can be a thioether linker. An example of a thioether linker is, but is not limited to:

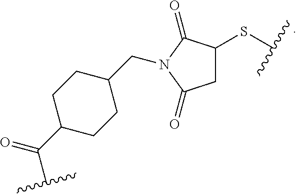 [SMILES: *SC1CC(=O)N(CC2CCC(C(*)=O)CC2)C1=O]

In a further aspect, the linker can be a dipeptide linker. An example of a dipeptide linker is, but is not limited to:

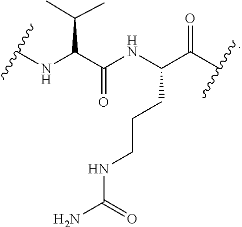

In a further aspect, the linker can be a maleimidocaproyl linker. An example of a maleimidocaproyl linker is, but is not limited to:

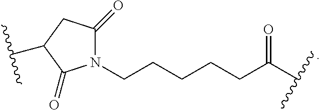 [SMILES: *C(=O)CCCCCN1C(=O)CC(*)C1=O]

In a further aspect, the linker can be a hydrazone. Examples of hydrazone linkers include, but are not limited to:

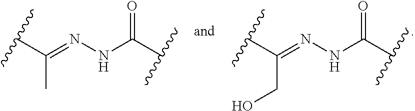

In a further aspect, the linker can be selected from —NR⁶¹C(O)—, —C(O)NR⁶¹—, —NR⁶¹C(S)NR⁶²—, —SCH₂C(O)—, —C(O)SCH₂—,

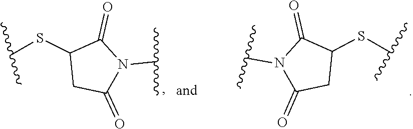 [SMILES: *SC1CC(=O)N(*)C1=O ; *SC1CC(=O)N(*)C1=O]

In a further aspect, v is selected from

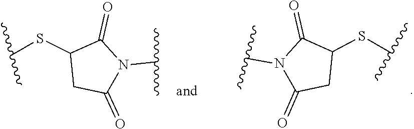 [SMILES: *SC1CC(=O)N(*)C1=O ; *SC1CC(=O)N(*)C1=O]

In a still further aspect, the linker is

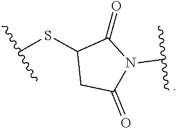 [SMILES: *SC1CC(=O)N(*)C1=O]

In yet a further aspect, the linker is

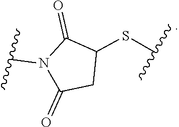 [SMILES: *SC1CC(=O)N(*)C1=O]

In a further aspect, the linker is selected from —NR⁶¹C(O)—, —C(O)NR⁶¹—, —NR⁶¹C(S)NR⁶²—, —SCH₂C(O)—, and —C(O)SCH₂—. In a still further aspect, the linker is selected from —NR⁶¹C(O)— and —C(O)NR⁶¹—In yet a further aspect, the linker is —NR⁶¹C(O)—. In an even further aspect, the linker is —C(O)NR⁶¹—.

In a further aspect, the linker can be selected from —NR⁶¹C(S)NR⁶²—, —SCH₂C(O)—, and —C(O)SCH₂—. In a still further aspect, the linker is selected from —SCH₂C(O)— and —C(O)SCH₂—. In yet a further aspect, L is —NR⁶¹C(S)NR⁶²—. In an even further aspect, the linker is —SCH₂C(O)—. In a still further aspect, the linker is —C(O)SCH₂—.

In a further aspect, the linker can be cucurbit[n]uril, polyethylene glycol, polyglycine, polyamides, polyesters, alkyl hydrocarbons, or aryl hydrocarbons.

The conjugates as described herein can also comprise a detectable label. For example, disclosed herein are molecular probes, comprising the disclosed conjugate. The phrase “detection label” as used herein refers to any molecule that can be associated with the compositions described herein, directly or indirectly, and which results in a measurable, detectable signal, either directly or indirectly. For instance, the label can be attached to one or more of the therapeutic peptides or polypeptides. In some aspects, the label can be attached to the Zwitterion polymer. In some aspects, a molecular probe comprising a conjugate described herein, further comprises a detectable label.

Examples of detectable labels include fluorescent, radioactive isotopes, fluorescent molecules, phosphorescent molecules, enzymes, antibodies, and ligands. Examples of fluorescent labels include, but are not limited to SYBR Green I (Invitrogen), fluorescein isothiocyanate (FITC), 5,6-carboxymethyl fluorescein, Texas red, nitrobenz-2-oxa-1,3-diazol-4-yl (NBD), coumarin, dansyl chloride, rhodamine, amino-methyl coumarin (AMCA), Eosin, Erythrosin, BODIPY®, Cascade Blue®, Oregon Green®, pyrene, lissamine, xanthenes, acridines, oxazines, phycoerythrin, macrocyclic chelates of lanthanide ions such as quantum dye′, fluorescent energy transfer dyes, such as thiazole orange-ethidium heterodimer, and the cyanine dyes Cy3, Cy3.5, Cy5, Cy5.5 and Cy7. Examples of other specific fluorescent labels include 3-Hydroxypyrene 5,8,10-Tri Sulfonic acid, 5-Hydroxy Tryptamine (5-HT), Acid Fuchsin, Alizarin Complexon, Alizarin Red, Allophycocyanin, Aminocoumarin, Anthroyl Stearate, Astrazon Brilliant Red 4G, Astrazon Orange R, Astrazon Red 6B, Astrazon Yellow 7 GLL, Atabrine, Auramine, Aurophosphine, Aurophosphine G, BAO 9 (Bisaminophenyloxadiazole), BCECF, Berberine Sulphate, Bisbenzamide, Blancophor FFG Solution, Blancophor SV, Bodipy F1, Brilliant Sulphoflavin FF, Calcien Blue, Calcium Green, Calcofluor RW Solution, Calcofluor White, Calcophor White ABT Solution, Calcophor White Standard Solution, Carbostyryl, Cascade Yellow, Catecholamine, Chinacrine, Coriphosphine O, Coumarin-Phalloidin, CY3.1 8, CY5.1 8, CY7, Dans (1-Dimethyl Amino Naphaline 5 Sulphonic Acid), Dansa (Diamino Naphtyl Sulphonic Acid), Dansyl NH—CH3, Diamino Phenyl Oxydiazole (DAO), Dimethylamino-5-Sulphonic acid, Dipyrrometheneboron Difluoride, Diphenyl Brilliant Flavine 7GFF, Dopamine, Erythrosin ITC, Euchrysin, FIF (Formaldehyde Induced Fluorescence), Flazo Orange, Fluo 3, Fluorescamine, Fura-2, Genacryl Brilliant Red B, Genacryl Brilliant Yellow 10GF, Genacryl Pink 3G, Genacryl Yellow SGF, Gloxalic Acid, Granular Blue, Haematoporphyrin, Indo-1, Intrawhite Cf Liquid, Leucophor PAF, Leucophor SF, Leucophor WS, Lissamine Rhodamine B200 (RD200), Lucifer Yellow CH, Lucifer Yellow VS, Magdala Red, Marina Blue, Maxilon Brilliant Flavin 10 GFF, Maxilon Brilliant Flavin 8 GFF, MPS (Methyl Green Pyronine Stilbene), Mithramycin, NBD Amine, Nitrobenzoxadidole, Noradrenaline, Nuclear Fast Red, Nuclear Yellow, Nylosan Brilliant Flavin EBG, Oxadiazole, Pacific Blue, Pararosaniline (Feulgen), Phorwite AR Solution, Phorwite BKL, Phorwite Rev, Phorwite RPA, Phosphine 3R, Phthalocyanine, Phycoerythrin R, Polyazaindacene Pontochrome Blue Black, Porphyrin, Primuline, Procion Yellow, Pyronine, Pyronine B, Pyrozal Brilliant Flavin 7GF, Quinacrine Mustard, Rhodamine 123, Rhodamine 5 GLD, Rhodamine 6G, Rhodamine B, Rhodamine B 200, Rhodamine B Extra, Rhodamine BB, Rhodamine BG, Rhodamine WT, Serotonin, Sevron Brilliant Red 2B, Sevron Brilliant Red 4G, Sevron Brilliant Red B, Sevron Orange, Sevron Yellow L, SITS (Primuline), SITS (Stilbene Isothiosulphonic acid), Stilbene, Snarf 1, sulpho Rhodamine B Can C, Sulpho Rhodamine G Extra, Tetracycline, Thiazine Red R, Thioflavin S, Thioflavin TCN, Thioflavin 5, Thiolyte, Thiozol Orange, Tinopol CBS, True Blue, Ultralite, Uranine B, Uvitex SFC, Xylene Orange, and XRITC. Fluorescent labels can be obtained from a variety of commercial sources, including Invitrogen, Carlsbad, CA; Amersham Pharmacia Biotech, Piscataway, NJ; Molecular Probes, Eugene, OR; and Research Organics, Cleveland, Ohio.

In some aspects, the disclosed conjugates can prolong the half-life of the linked therapeutic peptides or polypeptides until a need for activity occurs. In some aspects, the conjugation of the therapeutic peptides or polypeptides to the Zwitterion polymer as described herein can increase the half-life of the therapeutic peptides or polypeptides in vitro and/or in vivo. In some aspects, the therapeutic peptides or polypeptides can have an in vivo half-life in humans of at least 5 hours. For example, the half-life can be 5-10, 10-20, or 12-50 hours. In some aspects, the half-life can be 20 hours or longer. Because of its longer half-life the conjugate can be administered less frequently.

In some aspects, the Zwitterion polymer comprises a plurality of monomer units. In some aspects, the monomer units can be S-alkyl-L-methionine sulfonium chloride where the alkyl group contains a carboxylic acid. In some aspects, the at least one monomer unit comprising S-alkyl-L-methionine sulfonium chloride where the alkyl group contains a carboxylic acid can be between 12 and 320. In some aspects, the at least one monomer unit comprising S-alkyl-L-methionine sulfonium chloride where the alkyl group contains a carboxylic acid can be 12, 25, 80, 175, 320, or any number in between. In some aspects, the at least one monomer unit comprising S-alkyl-L-methionine sulfonium chloride where the alkyl group contains a carboxylic acid can be 80.

In some aspects, the conjugate can comprise

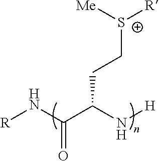

wherein R can be an alkyl group or an aryl group with or without a heteroatom; wherein R′ can be an alkyl group or an aryl group with or without a heteroatom or an alkyl group or an aryl group with or without a heteroatom containing a carboxylic acid group. In some aspects, n can be 10-400 or any number in between. In some aspects, n can be 12-320 or any number in between.

In some aspects, the conjugate can comprise:

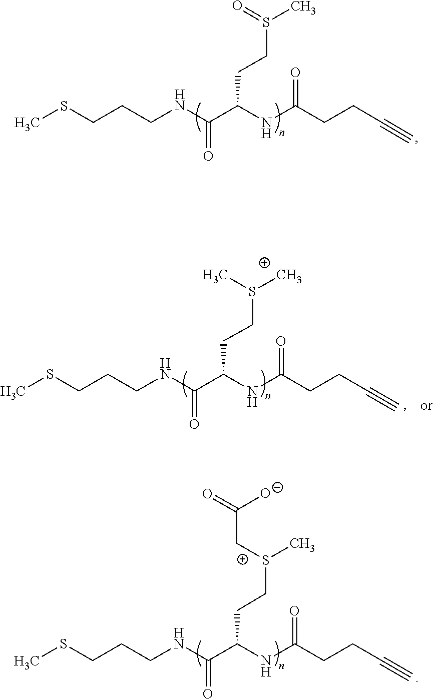

The methods disclosed herein related to the process of producing the conjugates as disclosed can be readily modified to produce a pharmaceutically acceptable salt of the conjugates. Pharmaceutical compositions including such salts and methods of administering them are accordingly within the scope of the present disclosure.

Also disclosed herein are pharmaceutical compositions comprising the disclosed conjugates.

Pharmaceutical Compositions

As disclosed herein, are pharmaceutical compositions, comprising the conjugates and a pharmaceutical acceptable carrier described herein. In some aspects, the e pharmaceutical composition can be formulated for intravenous administration. In some aspects, the pharmaceutical composition can be formulated for intravenous administration injection. The compositions of the present disclosure also contain a therapeutically effective amount of a conjugate comprising a therapeutic peptide or polypeptide as described herein. The pharmaceutical compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used to deliver the conjugates. Thus, compositions can be prepared for parenteral administration that includes conjugates dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like). Where the compositions are formulated for application to the skin or to a mucosal surface, one or more of the excipients can be a solvent or emulsifier for the formulation of a cream, an ointment, and the like.

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

Methods

Disclosed herein, are methods of treating a subject with a disease. In some aspects, the methods can comprise: (a) identifying a patient in need of treatment; and (b) administering to the subject a therapeutically effective amount of the pharmaceutical composition comprising a conjugate comprising a Zwitterion polymer, cucurbit[n]uril, and a therapeutic polypeptide and a pharmaceutically acceptable carrier.

In some aspects, the methods can comprise administering a therapeutic amount of any of the conjugates disclosed herein to a subject suffering from the disease. In some aspects, the conjugates can further comprise a pharmaceutically acceptable carrier.

In some aspects, the disease can be any disease that can be treated with a therapeutic peptide, protein, polypeptide, or antibody. In some aspects, the disease can by diabetes (e.g., Type I or Type II), cancer, osteoporosis, anemia, or autoimmune indications including but not limited to rheumatoid arthritis, multiple sclerosis, lupus, hypercholesterolemia, asthma, inflammatory bowel disease, an infection (caused by an infectious organisms, e.g., bacteria, viruses, fungi or parasites), a medication overdose, and allograft rejection. In some aspects, a subject with a medication overdose can be administered any of the conjugates disclosed herein, wherein the therapeutic peptide or polypeptide can be a reversal agent (e.g., drug reversal).

In some aspects, the therapeutic peptide or polypeptide can be insulin, calcitonin, erythropoietin, an antibody, or a chimeric antibody (e.g., regdanvimab, trastuzumab, pertuzumab, bevacizumab, rituximab, adalimumab, and Etanercept).

Dosages of the conjugates (or therapeutic peptides) can vary between wide limits, depending upon the disease or disorder to be treated, the age and condition of the individual to be treated, etc. and a physician will ultimately determine appropriate dosages to be used.

Amounts effective for this use can depend on the severity of the disease and the weight and general state and health of the subject. Suitable regimes for initial administration and booster administrations are typified by an initial administration followed by repeated doses at one or more hourly, daily, weekly, or monthly intervals by a subsequent administration. For example, a subject can receive one or more dose of the conjugate one or more times per week (e.g., 2, 3, 4, 5, 6, or 7 or more times per week).

This dosage may be repeated as often as appropriate. If side effects develop the amount and/or frequency of the dosage can be reduced, in accordance with normal clinical practice. In some aspects, the pharmaceutical composition can be administered once every one to thirty days.

The total effective amount of the conjugates in the pharmaceutical compositions disclosed herein can be administered to a mammal as a single dose, either as a bolus or by infusion over a relatively short period of time, or can be administered using a fractionated treatment protocol in which multiple doses are administered over a more prolonged period of time (e.g., a dose every 4-6, 8-12, 14-16, or 18-24 hours, or every 2-4 days, 1-2 weeks, or once a month). Alternatively, continuous intravenous infusions sufficient to maintain therapeutically effective concentrations in the blood are also within the scope of the present disclosure.

The therapeutically effective amount of the one or more of the therapeutic agents present within the compositions described herein and used in the methods as disclosed herein applied to mammals (e.g., humans) can be determined by one of ordinary skill in the art with consideration of individual differences in age, weight, and other general conditions (as mentioned above). Because the conjugates of the present disclosure can be stable in serum and the bloodstream and in some cases more specific, the dosage of the conjugates including any individual component can be lower (or higher) than an effective dose of any of the individual components when unbound. Accordingly, in some aspects, the therapeutic peptides administered have increased efficacy or reduced side effects when administered as part of the conjugate as compared to when the therapeutic peptide is administered alone or not as part of a conjugate.

In some aspects, the pharmaceutical composition can be administered with another pharmaceutically active agent.

The pharmaceutical compositions disclosed herein can be administered alone or in conjunction with other compounds, such as therapeutic compounds or molecules, e.g. anti-inflammatory drugs, analgesics or antibiotics. Such administration with other compounds can be simultaneous, separate or sequential. The components can be prepared in the form of a kit which may comprise instructions as appropriate. In some aspects, the pharmaceutical compositions disclosed herein and the other therapeutic compound are directly administered to a patient in need thereof.

The invention also provides a kit of parts comprising a pharmaceutical composition of invention, and an administration vehicle including, but not limited to, capsules for oral administration, inhalers for lung administration and injectable solutions for intravenous administration.

The pharmaceutical compositions described above can be formulated to include a therapeutically effective amount of the disclosed conjugate. Therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to a type of disease or disorder (e.g., cancer).

The pharmaceutical compositions described herein can be administered to the subject (e.g., a human patient) in an amount sufficient to delay, reduce, or preferably prevent the onset of clinical disease. Accordingly, in some aspects, the subject can be a human subject. In therapeutic applications, compositions can be administered to a subject (e.g., a human patient) already with or diagnosed with a disease or disorder (e.g., diabetes (Type I or Type II), cancer) in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a pharmaceutical composition can be an amount that achieves a cure, but that outcome is only one among several that can be achieved. As noted, a therapeutically effective amount includes amounts that provide a treatment in which the onset or progression of the diabetes (or cancer) is delayed, hindered, or prevented, or the diabetes (or cancer) or a symptom of the diabetes (or cancer) is ameliorated. One or more of the symptoms can be less severe. Recovery can be accelerated in an individual who has been treated.

In some aspects, the methods of treatment disclosed herein can also include the administration of a therapeutically effective amount of radiation therapy, immunotherapy, chemotherapy, stem cell transplantation or a combination thereof.

Also disclosed herein are methods of preventing aggregation of a therapeutic polypeptide. In some aspects, the methods can comprise: conjugating a compound to a therapeutic polypeptide. In some aspects, the compound can comprise a Zwitterion polymer. In some aspects, the Zwitterion polymer can comprise one or more monomer units of S-alkyl-L-methionine sulfonium chloride where the alkyl group contains a carboxylic acid and a linker. In some aspects, the linker can be cucurbit[n]uril. In some aspects, the Zwitterion polymer can be covalently bonded to the linker. In some aspects, the linker can be covalently bonded to the therapeutic polypeptide. In some aspects, the linker can be non-covalently bonded to the therapeutic polypeptide.

In some aspects, the therapeutic peptide or polypeptide can be insulin, calcitonin, erythropoietin, an antibody, or a chimeric antibody (e.g., regdanvimab, trastuzumab, pertuzumab, bevacizumab, rituximab, adalimumab, and Etanercept).

In some aspects, the linker can be covalently or non-covalently bonded to an amino group, a hydroxyl group, a sulfhydryl group or a carboxyl group of the therapeutic polypeptide. In some aspects, the linker can be cucurbit[n]uril. In some aspects, the cucurbit[n]uril can be cucurbit[7]uril. In some aspects, the at least one monomer unit comprising Zwitterion polymer portion of the conjugate has a peak degree of polymerization between 12-320.

In some aspects, wherein the linker can be cucurbit[7]uril and the therapeutic polypeptide can be insulin.

Kits

The kits can include a composition or conjugate comprising a Zwitterion polymer, a linker, and a therapeutic polypeptide; and suitable instructions (e.g., written and/or audio-, visual-, or audiovisual material). In some aspects, the composition can further comprise a pharmaceutically acceptable carrier. In some aspects, the kits can include a pharmaceutical composition as described herein that is packaged together with instructions for use. The kits can also include one or more of the following: diluents, sterile fluid, syringes, a sterile container, gloves, vials or other containers, pipettes, needles and the like.

### EXAMPLES

### Example 1: Supramolecular Protein Stabilization with Zwitterionic Polypeptide-Cucurbit[7]Uril Conjugates

Instrumentation and general methods. Reactions were conducted under an inert atmosphere of N2, using oven-dried glassware unless otherwise stated. Hexanes and dichloromethane were purified by first purging with dry nitrogen, followed by passage through columns of activated 3 Å molecular sieves, while purged. THF was purified by passage through columns of activated alumina. Glassware was oven dried at 120° C. Infrared spectra were recorded on a Bruker Alpha ATR-FTIR Spectrophotometer. Deionized water (18 MΩ-cm) was obtained by passing in-house deionized water through a Thermo Scientific MicroPure UV/UF purification unit. Tandem gel permeation chromatography/light scattering (GPC/LS) was performed on an Agilent 1260 Infinity liquid chromatograph pump equipped with a Wyatt DAWN HELEOS-II light scattering (LS) and Wyatt Optilab T-rEX refractive index (RI) detectors. CD measurements of the polypeptide solutions were recorded in quartz cells with a path length of 0.1 cm, on a JASCO J-1500 CD spectrophotometer. A Tecan Infinite M200 plate reader was used for absorbance assays.

THF was purified by first purging with dry nitrogen, followed by passage through columns of activated alumina. The polymerizations were monitored for completion via ATR-FTIR. Separations were achieved using 10⁵, 10⁴, and 10³ Å Phenomenex Phenogel 5 μm columns using 0.10 M LiBr in DMF as the eluent at 60° C. The GPC/LS samples were prepared at concentrations of 3 mg/mL. ¹H NMR spectra were recorded on a Varian Mercury spectrometer (400 MHz) or an Agilent DirectDrive spectrometer (500 MHz) and are reported relative to deuterated solvent. Data for ¹H NMR are reported as follows: chemical shift (δ ppm), multiplicity, coupling constant (Hz) and integration. Data for ¹³C NMR spectra are reported in chemical shift. Peptide therapeutics were prepared (Meudom, R.; et al. Curr. Res. Chem. Biol. 2022, 2, 100013).

Experimental procedures. Synthesis of PMet Panel.

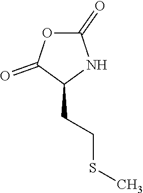

### L-Methionine-N-Carboxyanhydride (Met NCA)

Met NCA was prepared according to a published procedure, with minor modifications (Kramer, J. R. and Deming, T. J. Biomacromolecules 2010, 11 (12), 3668-3672). To a solution of L-methionine (1.00 g, 6.7 mmol) in dry THF (0.15 M) in a Schlenk flask was added a solution of phosgene in toluene (13.4 mmol, 20% (w/v), 2 equiv) via syringe. Phosgene is extremely hazardous and the manipulations are performed in a well-ventilated chemical fume hood with proper personal protection and the appropriate precautions taken to avoid exposure. The reaction was stirred under N₂ at 50° C. for 3 hrs, then evaporated to dryness and transferred to a dinitrogen filled glove box. The condensate in the vacuum traps was treated with 50 mL of concentrated aqueous NH₄OH to neutralize residual phosgene. Crude Met NCA, a yellow oil, was purified by anhydrous column chromatography (Wang, W. Int. J. Pharm. 2005, 289 (1-2), 1-30) in 20% THF in hexanes to give 2.11 g (91%) of the product as a colorless viscous liquid that spontaneously crystallized upon standing.

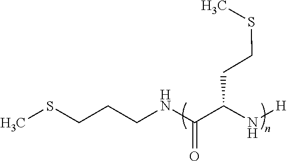

### Poly(L-Methionine), PMet, General Procedure for Polymerization of Met NCA

PMet was prepared (Kramer, J. R. and Deming, T. J. Biomacromolecules 2012, 13 (6), 1719-1723). Briefly, the polymerization reactions were performed in a dinitrogen filled glove box. To a solution of Met NCA in dry THF or DMF (50 mg/mL) was rapidly added, via syringe, a solution of (PMe₃)₄Co in dry THF (20 mM). The reaction was stirred at room temperature and polymerization progress was monitored by removing small aliquots for analysis by FTIR. Polymerization reactions were generally complete within 1 hour. Aliquots were removed for molecular weight analysis by endgroup analysis (vide infra). Reactions were removed from the glovebox and the polypeptide was precipitated into 1 mM aqueous HCl, >10× the reaction volume. PMet was collected by centrifugation. The white precipitate was washed with two portions of DI water and then lyophilized to yield PMet as a fluffy white solid in quantitative yield.

TABLE 1
Polymer information for structures used in this study.
M:I is the ratio of Met NCA to (PMe₃)₄Co. The polymer
reactions were conducted in THE, with the exception
of the 12 mer, which was prepared in DMF. Mₙ is the
number average molecular weight and DP = number
average degree of polymerization of PMet.

| M:I | Mₙ | DP |
|---|---|---|
| 4 | 1,574 | 12 |
| 8 | 3,280 | 25 |
| 25 | 10,496 | 80 |
| 60 | 22,960 | 175 |
| 100 | 41,984 | 320 |

pMet Molecular Weight Determination by Endgroup Analysis Via Endcapping with Poly(Ethylene Glycol)

PMet molecular weight was determined (Brzezinska, K. R.; et al. Macromolecules 2002, 35 (8), 2970-2976). Upon completion of the reaction, as confirmed by FTIR, aliquots of PMet were removed. A solution of 1000 Da methoxy-isocyanoethyl-poly(ethylene glycol) (PEG-NCO) was added to the aliquots, (3 equiv per (PMe₃)₄Co). The reaction immediately turned from pale orange to green. The reaction was stirred overnight at room temperature. The solution was precipitated into 1 mM aqueous HCl, >10× the reaction volume. PEG-endcapped PMet was collected by centrifugation. The white solids were vortexed with water, then centrifuged 3 times to remove the unconjugated PEG. The PEG endcapped polymers were then isolated by lyophilization to yield white solids in quantitative yields. pMet-PEG was then reacted with bromoacetic acid to generate the water-soluble sulfonium salt (vide infra). To determine PMet molecular weights (Mₙ), ¹H NMR spectra were obtained. Integrations of methionine resonances versus the PEG resonance at δ 3.64 were used to obtain PMet lengths (see example in spectral data section). ¹H NMR (500 MHz, D₂O, 25° C.): δ 4.627 (br s, 1H), 4.269 (m, 2H), 3.760 (s, 0.747H) 3.59-3.41 (br m, 2H), 3.018 (br d, 3H), 2.455-2.39 (br d, 2H).

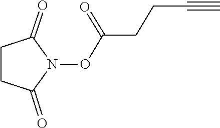

### Pentynoic Acid N-Hydroxysuccinide Ester

To a solution of pentynoic acid (1.00 g, 10.2 mmol) in dry THF (0.15 M) at 4° C. was added dicyclohexylcarbodiimide (2.208 g, 10.7 mmol), followed by N-hydroxysuccinimide (1.407 g, 12.2 mmol). The reaction was stirred under N₂ for 6 hrs at room temperature, sealed, and placed in a 4° C. fridge overnight. The DCU crystals were filtered off and the filtrate condensed. The crude NHS-ester was purified by column chromatography with hexanes and EtOAc to give 1.8 g (91%) of the product as white crystalline solid. ¹H NMR (500 MHz, D₂O, 25° C.): δ 2.049 (s, 1H), 2.569-2.639 (m, 2H), 2.845-2.902, (m, 6H).

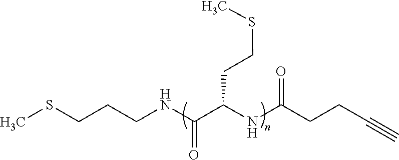

End-Capping of PMet with Pentynoic Acid N-Hydroxysuccinide Ester to Form PMet-Alkyne, PMet-Alk

PMet was dissolved in THF (20 mg/mL). 1 Equiv of NaHCO₃ and 5 equivs of pentynoic acid N-hydroxysuccinide ester per terminal amine group was added. The reaction was allowed to stir for 16 hrs at room temperature. PMet-Alk was used directly in the alkylation and oxidation reactions.

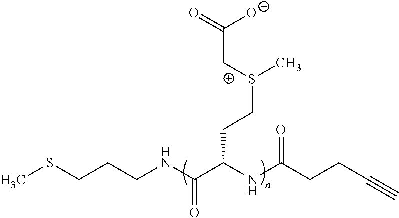

### Preparation of Poly-S-Carboxymethyl-L-Methionine Sulfonium Chloride Alkyne, PMet CM-Alk

To the solution of PMet-Alk in THF was added an equivalent volume of water. Bromoacetic acid (3 equivs per methionine residue) was added and the reaction was stirred at room temperature for 48 hours. The THF was removed by rotary evaporation and the reaction was transferred to a 2000 MWCO dialysis bag, and dialyzed against 0.10 M NaCl for 24 hours, followed by DI water for 48 hours with water changes twice per day. The contents of the dialysis bag were then lyophilized to dryness to give the product PMet^{CM}-Alk, as a white solid. Average yield is ˜85% with loss assumed to be due to the dialysis bag.

¹H NMR (500 MHz, D₂O, 25° C.): δ 4.617 (br s, 1H), 4.318-4.218 (br m, 2H), 3.449-3.513 (br m, 2H), 3.015-3.003 (br d, 3H), 2.48-2.317 (br d, 2H).

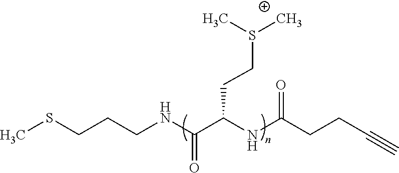

### Preparation of Alkyne-Poly-S-Methyl-L-Methionine Sulfonium Chloride, PMet^{M}-Alks

To the solution of PMet-Alk in THF was added an equivalent volume of water. Methyl iodide (3 equivs per methionine residue) was added, the reaction was covered with foil, and stirred at room temperature for 48 hours. The THF was removed by rotary evaporation and the reaction was transferred to a 2000 MWCO dialysis bag, and dialyzed against 0.10 M NaCl for 24 hours, followed by DI water for 48 hours with water changes twice per day. Dialysis against NaCl serves to exchange counterions from iodide to chloride. The contents of the dialysis bag were then lyophilized to dryness to give the product PMet^{M}-Alk, as a white solid. Average yield is ˜85% with loss assumed to be due to the dialysis bag. ¹H NMR (500 MHz, D₂O, 25° C.): δ 4.456 (br s, 1H), 3.314 (br s, 2H), 2.850-2.845 (br d, 6H), 2.24-2.127 (br d, 2H).

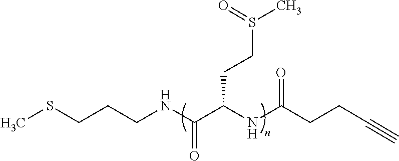

### Preparation of Poly(L-Methionine Sulfoxide)-Alkyne, PMet^{O}-Alks

Crude PMet-Alk from the NHS ester coupling reaction was suspended in 30% H₂O₂ with 1% AcOH at 20 mg/mL at 4° C. The heterogeneous reaction was stirred vigorously. After ca. 30 min, solids were observed to have dissolved to generate a homogenous solution. The reaction was stirred for a further 15 min. 1M Sodium thiosulfate was added dropwise until the evolution of bubbles ceased. The reaction was transferred to a 2000 MWCO dialysis bag, and dialyzed against 0.10 M NaCl for 24 hours, followed by DI water for 48 hours with water changes twice per day. The contents of the dialysis bag were then lyophilized to dryness to give the product PMet®-Alk, as a white solid. Average yield is ˜85% with loss assumed to be due to the dialysis bag. ¹H NMR (500 MHz, D₂O, 25° C.): δ 4.542 (br s, 1H), 3.048 (br m, 2H), 3.782 (br s, 3H), 2.240-2.328 (br d, 2H).

Procedure for click reaction to PMet-Alks. CB[7]-N₃ (19 mg) was synthesized (Vinciguerra, B.; et al. J. Am. Chem. Soc. 2012, 134 (31), 13133-13140), and attached to MetCM80-Alkyne via copper-catalyzed click methods (Zou, L.; et al. ACS Appl. Mater. Interfaces 2019, 11 (6), 5695-5700) Briefly, CB[7]-N₃ was combined along with PMet^{CM}₈₀-Alkyne (230 mg), copper(II) sulfate pentahydrate (CuSO4·5H2O, 0.18 mg) and PMDETA (98%, 0.6 μL) and dissolved in 10 mL water in a Schlenk flask. The flask was degassed with three freeze-pump-thaw cycles. On the last cycle, the flask was opened to quickly add sodium ascorbate (0.9 mg) into the flask before re-capping the flask. The flask was vacuumed and backfilled with N₂ for 5 cycles before immersion in a 50° C. oil bath to thaw the solution and initiate the ‘click’ reaction. After 2 days, the reaction was quenched by exposure to air. The reaction mixture was then transferred into dialysis tubing (MWCO=3500) and dialyzed against water for two days. The pure product was obtained after lyophilization as yellow solid and was determined by ¹H-NMR (400 MHz) to be fully substituted with CB[7].

Calcitonin Modification.

hCT (24 mg, 7.02 μmol) and NaBH 3 CN (5 equi., 35.1 μmol, 2.2 mg) were dissolved in 2.7 mL citric acid buffer (pH 6.1). 0.5 M tert-butyl 4-formylbenzylcarbamate in DMSO (2 equi., 14.04 μmol, 28 μL) or 0.5 M tert-butyl 4-formylphenylethylcarbamate in DMSO (2 equi., 14.04 μmol, 28 μL) was added into the system and stirred for 4 h at room temperature. The reaction solution was diluted with 1:1 acetonitrile/water (10.0 mL) and lyophilized. The lyophilized powder was deprotected in 1 mL solution of 95% TFA, 2.5% water and 2.5% TIS for 10 min. The solution was further diluted with 3 mL of water, purified via preparative HPLC and lyophilized to provide pure hCT A1 and hCT A2 samples.

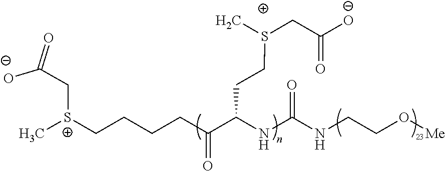

Fluorophore Conjugation to PMet^{CM}₈₀

PMet^{CM}₈₀ (2 mg) was dissolved in 0.2 mL MilliQ water. The solution was basified with 40 μL 5% NaHCO₃. A solution of AF350-NHS (45 μL, 5 g/L, 5 molar equiv. per polypeptide) was added and the reaction allowed to stand for 48 hours. Next, the reaction was diluted 3-fold with MilliQ water. The labeled polypeptide was purified using 3 kDa MWCO Amicon Ultra-2 spin filter three times. The resulting solution was lyophilized to yield an off-white foam (1.3 mg).

Circular dichroism. Polymers were dissolved in or milliQ water. Aliquots were taken and passed through a 0.45 μm filter before determining peptide concentration by UV-Vis spectrophotometry on a SpectraMax M2 spectrophotometer. A wavelength of 214 nm, extinction coefficient of 2200 cm⁻¹M⁻¹, and Beer's law were used to determine peptide concentration and normalize CD data. Samples were prepared at concentrations between 0.25 and 1 mg/mL. Spectra were recorded as an average of 3 scans. The molar ellipticity ([θ]) was calculated using the equation [θ]=(θ*100)/(c*1), where θ is measured ellipticity (mdeg), c is concentration (M), and 1 is path length of the cuvette (cm).

Aggregation assay methods. Insulin aggregation assay. Insulin aggregation was assessed (Sluzky, V.; et al. Proc. Natl. Acad. Sci. U.S.A 1991, 88 (21), 9377-9381; and Webber, M. J.; et al. Proc. Natl. Acad. Sci. U.S.A 2016, 113 (50), 14189-14194). Insulin or calcitonin samples at pH 7.4 PBS and a final concentration of 1 mg/mL were prepared with and without the addition of 1.5 molar equivs of CB [7]-PMet. Samples were plated in a clear 96-well plate (Thermo Scientific Nunc) at a volume of 150 μL per well (n=5 wells/group) and sealed with an optically clear and thermally stable seal. The plate was immediately placed into a Tecan Infinite M200 plate reader and shaken continuously at 37° C. Absorbance readings at 540 nm were collected every 6 min the duration of the experiment as reported, and absorbance values were subsequently converted to transmittance.

Calcitonin aggregation assay. hCT and analogs were dissolved in PBS buffer, pH 7.4 and centrifuged at 3000 rpm for 3 min. The supernatant concentration was determined by nanodrop (6=1615 M⁻¹cm⁻¹ at 280 nm). hCT and analogs were prepared at a final concentration of 0.5 mg/mL with and without 5 eq of PMet^{CM}₈₀-CB[7]. Samples were plated at 150 μL per well in a clear 96-well plate (Fisher Scientific Nunc) and sealed with optically clear and thermally stable sealing tape (VWR). The plate was immediately placed into the plate reader at 37° C. Absorbance readings at 540 nm were collected every 6 min with 60 s shaking between reads for 30 h. Absorbance values were subsequently converted to transmittance.

Cytotoxicity and biodegradation. Cellular viability assay. MDA-MB-231 cells were cultured in Dulbecco's Modified Eagle Medium with 10% fetal bovine serum, 2 mM L-glutamine, and 100 U/mL penicillin. Upon reaching sufficient confluency, cells were trypsinized and suspended in medium. Cells were loaded 5×10³ per well in a clear flat bottom 96-well plate. 24 hours after plating, cells were treated with varied amounts of polypeptide for 24 hours, then analyzed using a CCK-8 assay from Dojingo Molecular Technologies, Inc. The CCK-8 reagent was allowed to incubate with cells for 4 hours prior to absorbance reading at 450 nm.

Protease digestions. Digestions were performed with 40 μg of AF350-PMet^{CM}₈₀ at a final volume of 30 μL. 0.05% Gibco Trypsin was used at varied E:S ratios in a reaction buffer of 50 mM NH₄HCO₃ pH 8. Methionine aminopeptidase 2 (METAP2 from R&D Systems) was used at varied E:S ratios in a reaction buffer of 50 mM Hepes, 100 mM NaCl, mM CoCl₂ at pH 7.4. Protease K was obtained from ThermoFisher (#AM2542). Digestions with Proteinase K were performed in 1×PBS pH 7.4 with varied E:S ratios. Papain digestions were performed in McIlvaine buffer pH 6 and preincubated with glutathione for 5 minutes before adding polypeptide (Zhang, R.; et al. Macromol. Biosci. 2017, 17 (1), 1600125). Digestions were allowed to proceed for between 24 hours and 7 days at 37° C. sheltered from light.

Electrophoresis. Bis-tris 4-12% gels from BioRad were used. Digestions were diluted with 4× Loading Buffer from BioRad. 40 μg of polypeptide digestion were loaded into each lane. Gels ran at 175V for 40 minutes. Gels were visualized on a standard UV gel imager without any preparation after running.

Preparation of cucurbit[7]uril (CB[7]) functional poly(L-methionine)s (PMet). L-Methionine-N-carboxyanhydride, Met NCA, was prepared and polymerized according to a published procedure, with minor modifications (Kramer, J. R.; Deming, T. J. Biomacromolecules 2010, 11 (12), 3668-3672). Molecular weights were determined by ¹H NMR end-group analysis (Kramer, J. R.; Deming, T. J. Biomacromolecules 2010, 11 (12), 3668-3672). After polymerization, the polypeptides were precipitated into 1 mM aqueous HCl, collected by centrifugation and the precipitate was washed with two portions of DI water. In THF, the polypeptides (10 mg/mL) were reacted with 5 equivs of pentynoic acid N-hydroxysuccinide ester and 1 equiv of sodium bicarbonate for 16 h at ambient temperature. The solutions were split for alkylation or oxidation without further purification. For oxidation, the THF was removed and the polypeptide subjected to 30% H₂O₂ with 1% AcOH (20 mg/mL) at 4° C. for 45 minutes. 1M Sodium thiosulfate was then added dropwise until the evolution of bubbles ceased. For alkylation, an equal volume of water to THF was added to the THF-polypeptide solution from the previous step. Either bromoacetic acid or methyl iodide (3 equiv) was added, and the reaction stirred for 48 h. All polymers were transferred to 2000 MWCO dialysis tubing and dialyzed first against 0.10M NaCl, then Milli-Q water for 48 h, and finally lyophilized. For CB[7] functionalization, CB[7]-N₃ was synthesized and attached to PMet^{CM}₈₀-Alk via copper-catalyzed click chemistry (Zou, L.; et al. ACS Appl. Mater. Interfaces 2019, 11 (6), 5695-5700). The final product was transferred into dialysis tubing (MWCO=3500) and dialyzed against Milli-Q water for two days and lyophilized to dryness.

Aggregation assays. Peptide therapeutics were prepared (Meudom, R.; et al. Curr. Res. Chem. Biol. 2022, 2, 100013) and aggregation was assessed (Sluzky, V.; et al. Proc. Natl. Acad. Sci. U.S.A 1991, 88 (21), 9377-938; and Webber, M. J.; et al. Proc. Natl. Acad. Sci. U.S.A 2016, 113 (50), 14189-14194). Insulin or calcitonin solutions were prepared in PBS at pH 7.4 at a final concentration of 1 mg/mL, and with and without the addition of 1.5 molar equivs of CB [7]-PMet for rHu insulin and 5 molar equivs for hCT. Samples were plated in a clear 96-well plate (Thermo Scientific Nunc) at a volume of 150 μL per well (n=wells/group) and sealed with an optically clear and thermally stable seal. The plate was shaken continuously at 37° C. and absorbance readings at 540 nm were collected every 6 min the duration of the experiment as reported. Absorbance values were subsequently converted to transmittance.

Calcitonin modification. hCT (24 mg, 7.02 μmol) and NaBH₃CN (5 equivs, 35.1 μmol, 2.2 mg) were dissolved in 2.7 mL citric acid buffer (pH 6.1). 0.5 M Tert-butyl 4-formylbenzylcarbamate in DMSO (2 equivs, 14.04 μmol, 28 μL) or 0.5 M tert-butyl 4-formylphenylethylcarbamate in DMSO (2 equivs, 14.04 μmol, 28 μL) was added into the system and stirred for 4 h at room temperature. The reaction solution was diluted with 1:1 acetonitrile/water (10.0 mL) and lyophilized. The lyophilized powder was deprotected in 1 mL of a solution of 95% trifluoroacetic acid, 2.5% water and 2.5% triisopropylsilane for 10 min. The solution was further diluted with 3 mL of water, purified via preparative HPLC and lyophilized to provide pure hCT A1 and hCT A2 samples.

Cellular viability. MDA-MB-231 cells were cultured in Dulbecco's Modified Eagle Medium with 10% fetal bovine serum, 2 mM L-glutamine, and 100 U/mL penicillin. Upon reaching sufficient confluency, cells were trypsinized and suspended in medium. Cells were loaded 5×103 per well in a clear flat bottom 96-well plate. 24 hours after plating, cells were treated with varied amounts of polypeptide for 24 hours, then analyzed using a CCK-8 assay from Dojingo Molecular Technologies, Inc. The CCK-8 reagent was allowed to incubate with cells for 4 hours prior to absorbance reading at 450 nm.

Protease degradation. Digestions were performed with 40 μg of AF350-PMetCM80 at a final volume of 30 μL. 0.05% Gibco Trypsin was used at varied E:S ratios in a reaction buffer of 50 mM NH₄HCO₃ pH 8. Methionine aminopeptidase 2 (METAP2 from R&D Systems) was used at varied E:S ratios in a reaction buffer of 50 mM Hepes, 100 mM NaCl, mM CoCl₂ at pH 7.4. Protease K was obtained from ThermoFisher (#AM2542). Digestions with Proteinase K were performed in 1×PBS pH 7.4 with varied E:S ratios. Papain digestions were performed in McIlvaine buffer pH 6 and preincubated with glutathione for 5 minutes before adding polypeptide. Digestions were allowed to proceed for between 24 hours and 7 days at 37° C. sheltered from light.

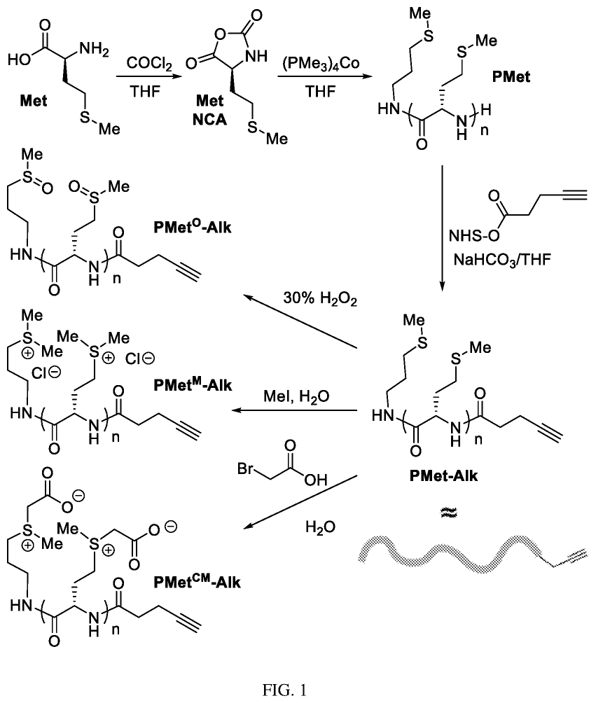

Results and discussion. To investigate the use of PMet as the polymeric component of a supramolecular protein stabilization reagent, a panel of PMet chain lengths ranging from 12 to 320 residues were prepared. These were generated via polymerization of Met N-carboxyanhydride (NCA) (FIG. 1; scheme 1). Using previously reported methods, Met NCA was prepared from Met on multi-gram scale in one step. Subsequent metal-catalyzed polymerizations to yield PMet were typically complete in <1 h and in quantitative yield. PMet was easily separated from the catalyst by precipitation into acidic water and was collected as a white powder.

The PMet amino-terminal was functionalized with an alkyne moiety (PMet-Alk) via an NHS ester compound prepared from commercially available pentynoic acid. In the same pot, PMet-Alk was then either oxidized or alkylated. To generate neutral PMetO-Alk, PMet-Alk was treated with H₂O₂ for 45 min at 4° C. (Scheme 1; FIG. 1). Alternatively, PMet-Alk was treated with 3 equivalents of methyl iodide or bromo-acetic acid for 16 h at ambient temperature to generate the cationic methyl or zwitterionic carboxymethyl sulfonium salts PMetM-Alk and PMetCM-Alk, respectively (Scheme 1; FIG. 1). The polypeptide structures were readily soluble in water. PMetM-Alk was selected as a cationic comparison to neutral PMetO-Alk and zwitterionic PMetCM-Alk. The PMet-Alk species were purified by dialysis against NaCl which served to exchange counterions to chloride, followed by MilliQ water. After lyophilization, ¹H NMR data confirmed the conversions were quantitative.

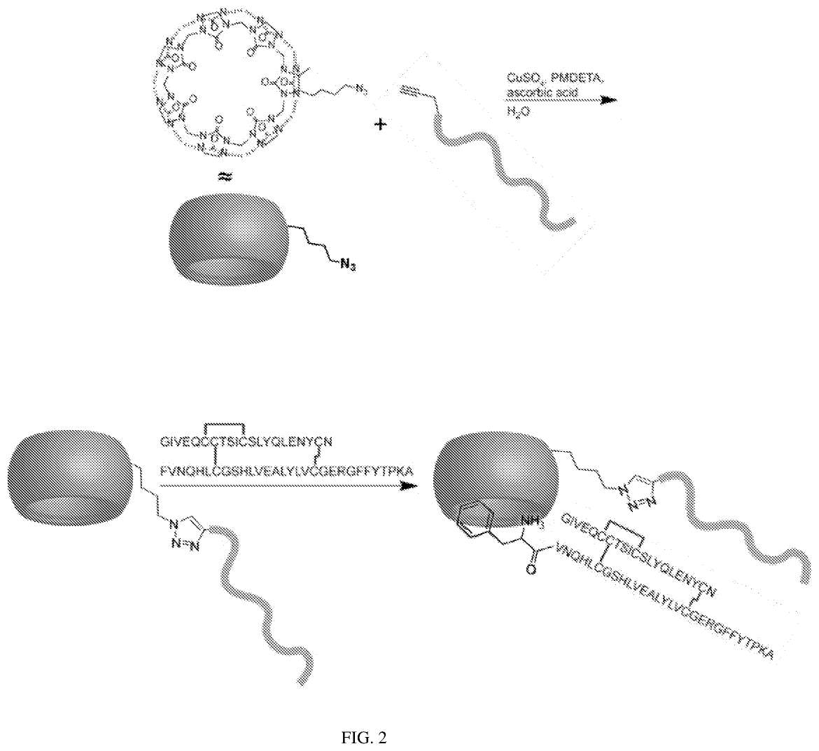

The azide-bearing monofunctional macrocycle, CB[7]-N₃, was synthesized (Vinciguerra, B.; et al. J. Am. Chem. Soc. 2012, 134 (31), 13133-13140). Subsequently, PMet-Alks of varied structure were covalently conjugated to CB[7]-N₃ via copper-catalyzed click reactions utilizing copper(II) sulfate pentahydrate, sodium ascorbate, and N,N,N′,N″,N″-pentamethyldiethylenetriamine (PMDETA) (Scheme 2; FIG. 2) (Zou, L.; et al. ACS Appl. Mater. Interfaces 2019, 11 (6), 5695-5700). Reaction products were purified by dialysis and then lyophilized. Analysis by ¹H-NMR indicated quantitative end-group modification and generation of the CB [7]-PMet panel.

Aggregation of recombinant human (rHu) insulin was assessed (Sluzky, V.; et al. Proc. Natl. Acad. Sci. U.S.A 1991, 88 (21), 9377-938; and Webber, M. J.; et al. Proc. Natl. Acad. Sci. U.S.A 2016, 113 (50), 14189-14194). Briefly, 1 mg/mL insulin solutions were prepared in phosphate buffered saline (PBS) buffer at pH 7.4, and with or without the addition of 1.5 molar equivs of CB[7]-PMets (Webber, M. J.; et al. Proc. Natl. Acad. Sci. U.S.A. 2016, 113 (50), 14189-14194). Samples were plated and sealed, and then agitated continuously at 37° C. Absorbance readings at 540 nm were collected every 6 min for 100 h. This wavelength was selected since it is removed from the typical absorbance of both protein and polymer, enabling protein aggregation to be monitored by light scattering and a concomitant reduction of sample transmittance.

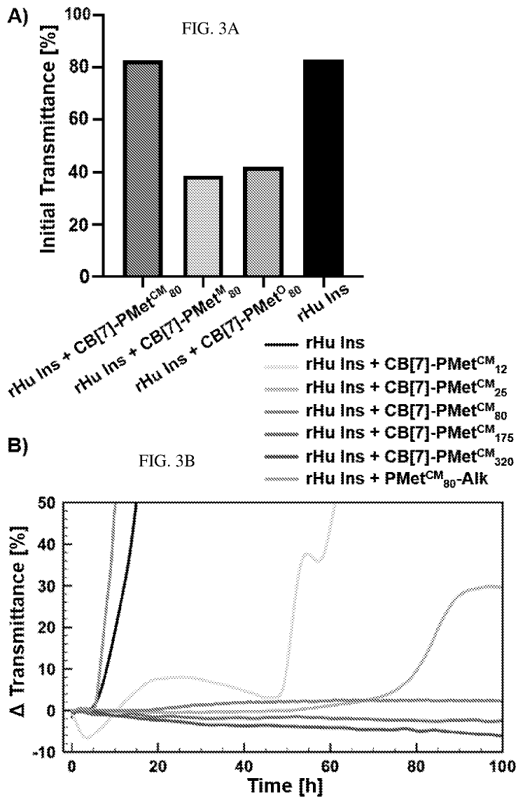

Medium chain lengths, 80mers, of CB[7]-PMets were examined in combination with rHu insulin. Solutions containing insulin with CB[7]-PMetO80 and CB [7]-PMetM80 became instantly turbid upon combination (FIG. 3A). However, CB[7]-PMetCM80 maintained the same initial transmittance as that of pure rHu insulin, confirming for-mulation solubility. Based on this data, the full panel of chain lengths were examined for CB[7]-PMetCM for their ability to inhibit insulin aggregation under stressed conditions.

Under continuous agitation at 37° C., it was observed that rHu insulin alone underwent rapid aggregation resulting in a substantial change in transmittance within the first few hours of the experiment. By contrast, rHu insulin in complex with CB[7]-PMetCM of chain lengths 175, or 320 was stable with no evidence of aggregation over the 100 h period of agitation (FIG. 3B). Shorter chain lengths of PMetCM were less successful at preventing aggregation. The 25mer inhibited aggregation until ca. 50 h, while the 12mer offered even less stabilization. The effect was due to supramolecular modification of insulin with PMetCM via the host-guest recognition, since the same polymer without the conjugated CB[7] macrocycle (PMetCM-Alk) offered no inhibition of insulin aggregation. CB[7] alone has already been shown to have no effect on rHu insulin stability.

The results show that differing solubilities of formulations from the neutral, cationic, and zwitterionic CB[7]-PMets can be rationalized by several considerations. The limited solubility of the cationic CB[7]-PMet^{M}₈₀ formulation is perhaps understood in the context of the clinically used insulin product, neutral protamine Hagedorn (NPH)-insulin. NPH-insulin relies on electrostatic insulin aggregation from formulation with the cationic protein protamine. Similarly, cationic PMet^{M} could form electrostatic complexes with anionic residues on insulin.

These results, however, were surprising in the stark differences in rHu insulin formulations of neutral CB[7]-PMet^{O}₈₀ and zwitterionic CB[7]-PMet^{CM}₈₀. Factors resulting in the difference between PMet^{O} and PMet^{CM} may result from the DMSO-like properties of PMet^{O}. Structural studies of DMSO-dissolved insulin indicated that the protein takes on multiple conformations including polyproline II-type helices, disordered structures, and α-helices, which may result in insoluble material.

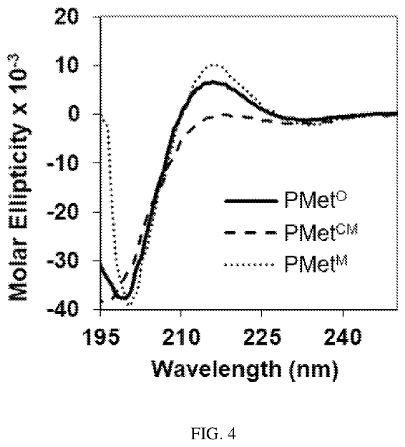

The differing formulation solubilities of the PMets are not likely ascribed to differences in their chain conformations. The secondary structures were analyzed by circular dichroism (CD) spectroscopy and PMet^{O}, PMet^{M}, and PMet^{CM} 80mers were found to yield very similar patterns indicative of disordered morphologies (FIG. 4). Since the effects can't be ascribed to chain conformation, it was tested whether differing hydrogen bonding (H-bonding) and water ordering properties play a role in insulin solvation. The sulfoxide structure takes on significant dipolar character, and studies of DMSO-H₂O interactions indicate one DMSO molecule forms two H-bonds, that the bonds are longer lived than water-water H-bonds, and that this induces linear ordering of water. By contrast, spectroscopic and modeling data indicate that zwitterionic ammonium betaine polymers homologous to PMet^{CM} do not alter the structure of the H-bonded network of water molecules. Water molecules at the polymer-material interface will be less oriented and similar to that of bulk water, which can influence insulin H-bonding.

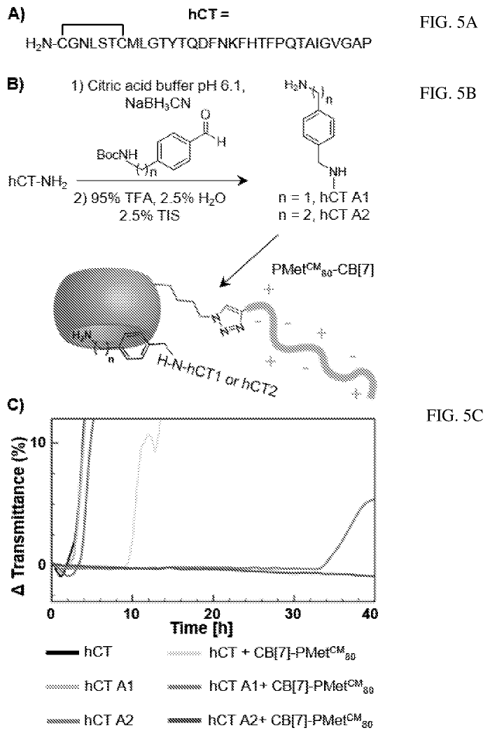

Next, the stabilizing effects of CB[7]-PMet^{CM}₈₀ on other aggregation-prone proteins was assessed. Human calcitonin (hCT, FIG. 5A) was selected as a model protein therapeutic. Since hCT lacks a terminal Phe for host-guest complexation, the N-terminal amine was selectively modified on-resin with a benzylic amine group using reductive amination chemistry (FIG. 5B). To optimize binding, two spacer lengths between the terminal amine and the benzyl ring were examined comprising either one or two methylene units (hCT A1 and A2, respectively). Aggregation behavior over 40 h was examined using the methods described herein, and with or without CB[7]-PMet^{CM}₈₀. Alone, hCT, hCT A1, and hCT A2 underwent rapid aggregation as noted by a rapid increase in transmittance (FIG. 5C). However, addition of CB[7]-PMet^{CM}₈₀ stabilized hCT A2 for at least 40 h with agitation, while hCT A1 was stabilized for ca. 34 h. Unmodified hCT lacking the terminal aromatic group was not stabilized by CB[7]-PMet^{CM}₈₀, indicating that supramolecular recognition of the protein by the CB[7] macrocycle is necessary to endow the protein with the anti-aggregation effects of the polymer.

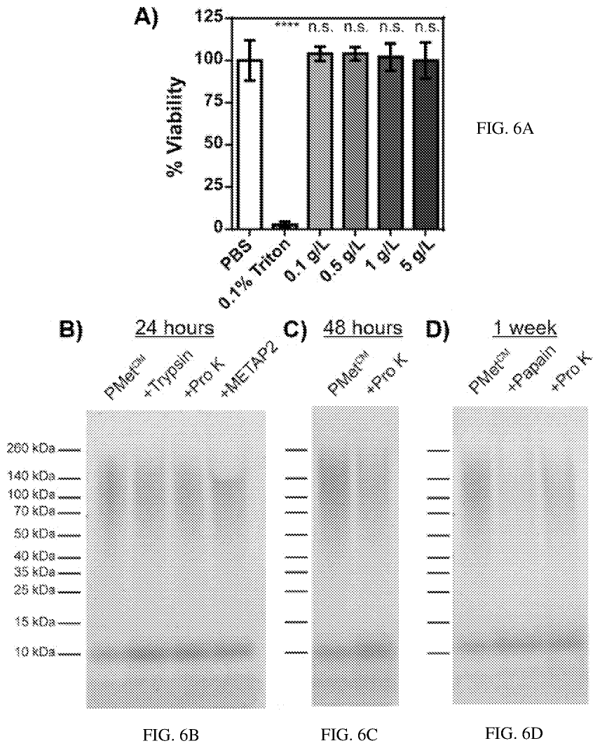

Since insulin is often dosed by diabetic people multiple times each day over the course of a lifetime, it is important for formulation additives to be non-toxic and readily degraded and/or cleared. Therefore, protease susceptibility and cytotoxicity properties of the lead anti-aggregation polymer, PMet^{CM}₈₀ were investigated. We analyzed the cytocompatibility properties using a commercial CCK-8 assay and human epithelial cell line MDA-MB-231. A broad concentration range from 0.1 g/L to 5 g/L, and after a 24-hour incubation period, PMet^{CM}₈₀ exhibited no statistically significant effect on cell viability at the concentrations studied (FIG. 6A). CB[7] has an IC₅₀ value of 0.53±0.02 mM in Chinese hamster ovary cells, and is tolerated in mice at up to 250 mg kg⁻¹ intravenous or 600 mg kg⁻¹ oral. The data demonstrate that the polypeptides will have low immunogenicity in vivo since a variety of zwitterionic polymers have avoided unwanted immune reactions and conjugates have dampened the response to known immunogenic proteins, and, thus, this formulation will have excellent biocompatibility properties.

Next, biodegradation of the PMet^{CM}₈₀ polypeptide polymer was evaluated by four different proteases: trypsin, methionine aminopeptidase 2 (MetAP2), proteinase K, and papain. Selected data is shown in FIGS. 6B-D. Protease digestion of AF350-labeled PMetCM80 visualized on SDS-PAGE gels over 48 hours with E:S of 1:5 for trypsin and proteinase K and 1:10 for METAP2. Trypsin cleaves the peptide bond between the carboxyl group of arginine or lysine and the amino group of the adjacent amino acid, so no degradation was expected. MetAP2 catalyzes the hydrolytic removal of N-terminal methionine residues, so it was tested whether the alkylated residues could be recognized despite the modification to the Met group. Proteinase K and papain was chosen as two broad spectrum, non-specific proteases that may be promiscuous enough to digest Met^{CM} residues. Proteinase K (Pro K) is an endogenous serine protease in humans and papain is a cysteine protease with similar activity to human cathepsins.

Since zwitterionic PMet^{CM}₈₀ was resistant to many common staining methodologies, it was end-functionalized with AF350-NHS to allow for in-gel visualization. After 24 h treatment with trypsin, proteinase K, or MetAP2, the polypeptide fluorescent signal was minimally decreased, suggesting negligible degradation (FIG. 6B). Therefore, Pro K and papain were chosen for further studies. Increasing both the incubation time and enzyme: substrate (E:S) ratio led to a decrease in fluorophore signal in the 48 h Pro K digestion (FIG. 6C). Finally, examination of the degradation of PMet^{CM}₈₀ after 1 week revealed partial degradation of PMet^{CM}₈₀ by Pro K and near complete proteolytic degradation by papain (FIG. 6D). These data show that PMet^{CM}₈₀ will biodegrade and in vivo accumulation can be avoided.

Overall, conjugates of zwitterionic polypeptides with a supramolecular macrocycle were developed as described herein, and show their usefulness as formulation additives to inhibit the aggregation of protein therapeutics. The zwitterionic polymer structure, PMet^{CM}, is derived from inexpensive amino acids, and is readily synthesized via rapid and scalable NCA polymerization. Conversion of the amino acid Met to the zwitterionic sulfonium structure is simple and quantitative. Neutral sulfoxide polypeptides and cationic sulfonium salts were also examined, but these were not efficient at inhibiting protein aggregation in the cases examined. At polymer lengths of 80mer or greater, zwitterionic PMet^{CM} was efficient at preventing insulin aggregation, while shorter chain lengths had more limited impact. This zwitterionic polypeptide exhibited no cytotoxicity in a human cell line and was slowly degraded by non-specific natural proteases. The data show that the slow degradation rate of PMet^{CM} is highly advantageous since the limited degradation and poor tissue clearance of formulation additives are accompanying challenges alongside aggregation in the development and distribution of protein therapeutics.

What is claimed is:

1. A conjugate, comprising a Zwitterion polymer, a linker, and a therapeutic polypeptide.

2. The conjugate of claim 1, wherein the Zwitterion polymer comprises one or more monomer units of S-alkyl-L-methionine sulfonium chloride where the alkyl group contains a carboxylic acid.

3. The conjugate of claim 2, wherein the S-alkyl-L-methionine sulfonium chloride where the alkyl group contains a carboxylic acid is alkylated.

4. The conjugate of claim 1, wherein the Zwitterion polymer is covalently bonded to the linker.

5. The conjugate of claim 1, wherein the linker is cucurbit[n]uril, polyethylene glycol, polyglycine, polyamides, polyesters, alkyl hydrocarbons, or aryl hydrocarbons.

6. The conjugate of claim 1, wherein the linker is covalently or non-covalently bonded to an amino group, a hydroxyl group, a sulfhydryl group or a carboxyl group of the therapeutic polypeptide.

7. The conjugate of claim 5, wherein cucurbit[n]uril is cucurbit[n]uril.

8. The conjugate of claim 1, wherein the Zwitterion polymer portion of the conjugate has a peak degree of polymerization between 12-320.

9. The conjugate of claim 1, wherein the therapeutic polypeptide is insulin, calcitonin, an antibody, or a chimeric antibody.

10. The conjugate of claim 9, having an in vivo half-life in humans of at least 12-50 hours.

11. A conjugate comprising: 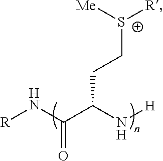
wherein R can be an alkyl group or an aryl group with or without a heteroatom; wherein R′ can be an alkyl group or an aryl group with or without a heteroatom or an alkyl group or an aryl group with or without a heteroatom containing a carboxylic acid group; and wherein n is 10-400.

12. A pharmaceutical composition comprising the conjugate of claim 1.

13. A method of treating a disease, the method comprising administering a therapeutic amount of the conjugate of claim 1 to a subject suffering from the disease.

14. The method of claim 13, wherein the disease is Type I diabetes, Type II, cancer, osteoporosis, anemia, rheumatoid arthritis, multiple sclerosis, lupus, hypercholesterolemia, asthma, inflammatory bowel disease, an infection, a medication overdose, or allograft rejection.

15. A method of preventing aggregation of a therapeutic polypeptide, the method comprising; conjugating a compound to a therapeutic polypeptide, wherein the compound comprises a Zwitterion polymer, wherein the Zwitterion polymer comprises one or more monomer units of S-alkyl-L-methionine sulfonium chloride where the alkyl group contains a carboxylic acid and linker, wherein the Zwitterion polymer is covalently bonded to the linker, and wherein the linker is covalently or non-covalently bonded to the therapeutic polypeptide.

16. The method of claim 15, wherein the therapeutic polypeptide is insulin, calcitonin, an antibody, or a chimeric antibody.

17. The method of claim 15, wherein the linker is covalently or non-covalently bonded to an amino group, a hydroxyl group, a sulfhydryl group or a carboxyl group of the therapeutic polypeptide.

18. The method of claim 15, wherein linker is cucurbit[7]uril.

19. The method of claim 15, wherein the linker is cucurbit[7]uril and the therapeutic polypeptide is insulin.


SEQUENCE LISTING (3 sequences)

SEQ ID NO: 1 - protein, 21 aa, synthetic construct
GIVEQCCTSICSLYQLENYCN

SEQ ID NO: 2 - protein, 30 aa, synthetic construct
FVNQHLCGSHLVEALYLVCGERGFFYTPKA

SEQ ID NO: 3 - protein, 32 aa, synthetic construct
CGNLSTCMLGTYTQDFNKFHTFPQTAIGVGAP